(19) United States
(12) Patent Application Publication
(10) Pub. No.: US 2005/0032052 A1
(43) Pub. Date: Feb. 10, 2005

(54) Methods and apparatus for extracting data from and optical analysis disc

(76) Inventors: Andrew Attila Pal, Rancho Santa Margarita, CA (US); Jesse James Morrow, Irvine, CA (US); Mark Oscar Worthington, Irvine, CA (US); Michael Craig Browne, Lake Forest, CA (US); Mikhail Matveev, Irvine, CA (US)

Correspondence Address: Donald Bollella, Esq.;BURSTEIN TECHNOLOGIES, Inc., 163 Technology Drive, Irvine, CA, 92618, US

(21) Appl. No.: 10/345,122

(22) Filed: Jan. 14, 2003

Related U.S. Application Data
(60) Provisional application No. 60/348,767, filed on Jan. 14, 2002.

(51) Int. Cl. G06F 17/60; C12Q 1/68; G06F 19/00; G01N 33/48; G01N 33/50

(52) U.S. Cl. 435/6; 702/20; 705/3

(57) ABSTRACT

Methods and apparatus for extracting data from an optical analysis disc such as a bio-disc are disclosed. The process of capturing the data is controlled by a single computer. In one embodiment, a template is provided in determining the location of desired sample regions. In another embodiment, one or more ditches are obliterated on the disc in determining the location of the desired samples. In one embodiment, a user enters measured distances to a control program to indicate the sample region location. The user may specify the sample rate. The user may also specify the number of capture regions. In one embodiment, capture regions are separated by radial lines. The head of a disc drive may be maintained at a constant radial distance. This distance is maintained to cause the disc to act as a centrifuge at a desired speed.

### RELATED APPLICATION INFORMATION

This application claims the benefit of priority from U.S. Provisional Patent Application Ser. No. 60/348,767 filed Jan. 14, 2002, the disclosure of which is hereby incorporated by reference in its entirety.

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND OF THE INVENTION

1. Field of the Invention

The present invention relates to the field of optical discs and, in particular, to methods and apparatus for extracting data from optical bio-discs.

2. Background Art Discussion

CDs and DVDs enable large amounts of data to be stored and quickly retrieved. Audio, visual, and computer program data are frequently stored on CDs or DVDs in a digital format. The end appearance of stored visual data is not readily apparent upon visual inspection. Thus, to extract visual information from a disc, a CD or DVD reader collects a stream of data that contains an encoding of the visual image. The functional aspects of a CD or DVD can be better understood with a review of CDs.

CDs and Related Optical Discs: A CD is a fairly simple piece of plastic, about four one-hundredths (4/100) of an inch (1.2 mm) thick. Most of a CD consists of an injection-molded piece of clear polycarbonate plastic. During manufacturing, this plastic is impressed with microscopic bumps arranged as a single, continuous, extremely long spiral track of data. Once the clear piece of polycarbonate is formed, a thin, reflective aluminum layer is sputtered onto the disc, covering the bumps. Then a thin acrylic layer is sprayed over the aluminum to protect it. The label is then printed onto the acrylic.

A CD has a single spiral track of data, circling from the inside of the disc to the outside. The fact that the spiral track starts at the center means that the CD can be smaller than 4.8 inches (12 cm) if desired. The data tracks are approximately 0.5 microns wide, with 1.6 microns separating one track from the next, and the elongated bumps that make up the track are each 0.5 microns wide, a minimum of 0.83 microns long and 125 nanometers high. Frequently, data is described as encoded in pits on a CD instead of bumps. Usually, the marks appear as pits on the aluminum side, but on the side the laser reads from, they are bumps. However, the scheme can be reversed.

The CD or Optical Disc Player: The CD player has the job of finding and reading the data stored on the CD. Considering how small the bumps are, the CD player is an exceptionally precise piece of equipment. The drive consists of three fundamental components. A drive motor spins the disc and is precisely controlled to rotate, typically, between 200 and 500 rpm depending on which track is being read. A laser and a lens system focus in on and read the bumps. A tracking mechanism moves the laser assembly so that the laser's beam can follow the spiral track. The tracking system has to be able to move the laser at micron resolutions.

Inside the CD player, there is frequently a degree of computer technology involved in forming the data into understandable data blocks and sending them either to the DAC (in the case of an audio CD) or to the computer (in the case of a CD-ROM drive). The fundamental job of the CD player is to focus the laser on the track of bumps. The laser beam passes through the polycarbonate layer, reflects off the aluminum layer and hits an opto-electronic device that detects changes in light. The bumps reflect light differently than the lands (the rest of the aluminum layer), and the opto-electronic sensor detects that change in reflectivity. The electronics in the drive interpret the changes in reflectivity in order to read the bits that make up the bytes.

One difficult task is keeping the laser beam centered on the data track. This centering is the job of the tracking system. The tracking system, as it plays the CD, has to continually move the laser outward. As the laser moves outward from the center of the disc, the bumps move past the laser faster. This happens because the linear, or tangential, speed of the bumps is equal to the radius times the speed at which the disc is revolving (rpm). Therefore, as the laser moves outward, the spindle motor must slow the speed of the CD. That way, the bumps travel past the laser at a constant speed, and the data comes off the disc at a constant rate.

### SUMMARY OF THE INVENTION

Embodiments of the present invention are directed to methods and apparatus for extracting data from an optical bio-disc. In one embodiment of the present invention, the process of extracting data from an optical bio-disc is controlled by an extraction system operation on a single computer that comprises an optical bio-drive. The extraction system comprises of two layers, an interface layer for receiving inputs from user governing the data capture and extracting process, and a control layer for controlling the operation of hardware components such as A/D cards and the optical bio-drive. In one embodiment, the control layer of the extraction system is written in an assembly language. Using programming shortcuts from the assembly language programming art, but previously not applied to bio-disc analysis systems, a single computer is capable of controlling the optical bio-drive, A/D card, and storage drive simultaneously.

The interface layer comprises two main interfaces. These include a centrifuge control interface and a capture program interface. The centrifuge control interface allows the user to spin the optical bio-disc at certain desired speed and duration to produce the effect of centrifugation. In one embodiment, the control layer of the extraction system causes the head of the optical bio-drive to be maintained at a constant radial distance.

The capture program interface can be accomplished using several embodiments. In one embodiment, several ditches are etched out on the disc and each one is positioned in front of a sampled area. One of the sample areas has a pair of leading ditches and is the primary trigger mark. The distance between the sample area and the ditch is fixed. The aim of the operation is to find the exact location of these sample areas using the ditches. According to one embodiment, the sampled space is a fixed size rectangular area. According to another embodiment, the sampled space is a fixed size circular area. According to yet another embodiment, the ditches may be binary encoded so that individual cells within the sampled area can be catalogued using the binary encoding as the address location of the individual cells.

The capture program interface, according to still yet another embodiment, allows a user to input parameters regarding the data capture process. The user can choose between time-based-capture and distance-based capture. In distance-based capture, a template is provided to assist in determining the location of one or more desired sample regions. The see-though template is placed over a bio-disc and markers on the template enable a user to determine a sample region's maximum and minimum radial distances on the bio-disc. In one embodiment, the user enters the measured distances as input to the control program. The program then calculates the correct moment to begin data capture using the input distances. In time-based capture, the user enters a beginning and end time rather than a distance to control when the data capture begins.

Besides choosing the capture option, the user can specify the sample rate in the capture program interface. The extraction system is configured to sample at the rate input by the user without the need of reprogramming. In one embodiment, the user can also specify the drive speed during capture. The optical drive can be set to spin at 1×, 2×, 4×, or any other desired rate obtainable in the subject drive. The user can also specify the number of capture regions that are to be captured. The software automatically de-interleaves the captured data into the correct number of data files (i.e., each of the sample areas corresponds to a separate data file).

The optical bio-disc embodiments of the present invention comprise physical features that enable the extraction system to conduct data capture in user-selected sample (capture) areas. First, capture areas may be designated by trigger markings on the outer rim of the bio-disc. In one embodiment, the markings are made by placing an opaque substance (e.g., silk screen indicia) on the outer rim. As the disc rotates, a first detector determines when the markings are present. When the markings are present, a second detector reads information from the disc. In one embodiment, the markings are also used to determine an ordering for the sample areas. In an example embodiment, the longest sequence of markings (e.g., the longest indicia area) is designated as the first sample area in the ordering. In another embodiment, the markings are part of an encoding scheme. In one embodiment, the markings encode the beginning of a sample area. In another embodiment, the markings encode the end of a sample area. In yet another embodiment, markings encode the size of the sample area. In still another embodiment, markings encode the sample area's position in the ordering of sample areas.

In one embodiment, capture regions are separated by radial lines. In one embodiment, the radial lines exist physically on the bio-disc. In an example embodiment, silk-screened “spokes” are used to form the lines. The regions are numbered starting at one of the radial lines and numbering either clockwise or counter-clockwise. In one embodiment, the thickest radial line or “spoke” is used to initiate numbering. In another embodiment, the thinnest radial line is used to initiate numbering. In yet another embodiment, the radial lines are logical. In one embodiment, the locations of the logical lines are determined relative to an identifiable sequence on the bio-disc. In another embodiment, a known region of the bio-disc encodes the location and number of sample regions.

In another embodiment of the capture program interface, the user can choose to have a stationary capture performed. In stationary capture, data is collected from the bio-disc while the head of the optical drive is maintained at a constant radial distance. As items (e.g., red blood cells) flow past the head position, the signal generated is altered. Thus, data is collected about changes in a region with respect to time. In one embodiment, the user can select to have the head positioned at one of eight pre-defined spots when the extraction system is reading an eight-sample area optical bio-disc.

In one embodiment, the behavior of the extraction system can be controlled using a scripting language. For example, the system can be scripted to centrifuge at a desired speed for a desired amount of time, incubate for another amount of time, and capture data from a desired region.

The present invention is directed to bio-discs, bio-drives, and in particular to hardware architecture of a bio-analyzer system including hardware implementations of cell counting methods. This invention or different aspects thereof may be readily implemented in, adapted to, or employed in combination with the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Biodiscs with Reflective Layers” filed Nov. 15, 2001; U.S. patent application Ser. No. 09/988,728 entitled “Methods and Apparatus for Detecting and Quantifying Lymphocytes with Optical Biodiscs” filed Nov. 20, 2001; U.S. patent application Ser. No. 09/988,850 entitled “Methods and Apparatus for Blood Typing with Optical Bio-discs” filed Nov. 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disk-Based Laboratory and Improved Optical Bio-Disc Including Same” filed Dec. 14, 2001; U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; U.S. patent application Ser. No. 10/038,297 entitled “Dual Bead Assays Including Covalent Linkages for Improved Specificity and Related Optical Analysis Discs” filed Jan. 4, 2002; U.S. patent application Ser. No. 10/043,688 entitled “Optical Disc Analysis System Including Related Methods for Biological and Medical imaging” filed Jan. 10, 2002; U.S. Provisional Application Ser. No. 60/348,767 entitled “Optical Disc Analysis System Including Related Signal Processing Methods and Software” filed Jan. 14, 2002 U.S. patent application Ser. No. 10/086,941 entitled “Methods for DNA Conjugation Onto Solid Phase Including Related Optical Biodiscs and Disc Drive Systems” filed Feb. 26, 2002; U.S. patent application Ser. No. 10/087,549 entitled “Methods for Decreasing Non-Specific Binding of Beads in Dual Bead Assays Including Related Optical Biodiscs and Disc Drive Systems” filed Feb. 28, 2002; U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” also filed Mar. 14, 2002; U.S. patent application Ser. No. 10/121,281 entitled “Multi-Parameter Assays Including Analysis Discs and Methods Relating Thereto” filed Apr. 11, 2002; U.S. patent application Ser. No. 10/150,575 entitled “Variable Sampling Control for Rendering Pixelization of Analysis Results in a Bio-Disc Assembly and Apparatus Relating Thereto” filed May 16, 2002; U.S. patent application Ser. No. 10/150,702 entitled “Surface Assembly For Immobilizing DNA Capture Probes in Genetic Assays Using Enzymatic Reactions to Generate Signals in Optical Bio-Discs and Methods Relating Thereto” filed May 17, 2002; U.S. patent application Ser. No. 10/194,418 entitled “Optical Disc System and Related Detecting and Decoding Methods for Analysis of Microscopic Structures” filed Jul. 12, 2002; U.S. patent application Ser. No. 10/194,396 entitled “Multi-Purpose Optical Analysis Disc for Conducting Assays and Various Reporting Agents for Use Therewith” also filed Jul. 12, 2002; U.S. patent application Ser. No. 10/199,973 entitled “Transmissive Optical Disc Assemblies for Performing Physical Measurements and Methods Relating Thereto” filed Jul. 19, 2002; U.S. patent application Ser. No. 10/201,591 entitled “Optical Analysis Disc and Related Drive Assembly for Performing Interactive Centrifugation” filed Jul. 22, 2002; U.S. patent application Ser. No. 10/205,011 entitled “Method and Apparatus for Bonded Fluidic Circuit for Optical Bio-Disc” filed Jul. 24, 2002; U.S. patent application Ser. No. 10/205,005 entitled “Magnetic Assisted Detection of Magnetic Beads Using Optical Disc Drives” also filed Jul. 24, 2002; U.S. patent application Ser. No. 10/230,959 entitled “Methods for Qualitative and Quantitative Analysis of Cells and Related Optical Bio-Disc Systems” filed Aug. 29, 2002; U.S. patent application Ser. No. 10/233,322 entitled “Capture Layer Assemblies for Cellular Assays Including Related Optical Analysis Discs and Methods” filed Aug. 30, 2002; U.S. patent application Ser. No. 10/236,857 entitled “Nuclear Morphology Based Identification and Quantification of White Blood Cell Types Using Optical Bio-Disc Systems” filed Sep. 6, 2002; U.S. patent application Ser. No. 10/241,512 entitled “Methods for Differential Cell Counts Including Related Apparatus and Software for Performing Same” filed Sep. 11, 2002; U.S. patent application Ser. No. 10/279,677 entitled “Segmented Area Detector for Biodrive and Methods Relating Thereto” filed Oct. 24, 2002; U.S. patent application Ser. No. 10/293,214 entitled “Optical Bio-Discs and Fluidic Circuits for Analysis of Cells and Methods Relating Thereto” filed on Nov. 13, 2002; U.S. patent application Ser. No. 10/298,263 entitled “Methods and Apparatus for Blood Typing with Optical Bio-Discs” filed on Nov. 15, 2002; and U.S. patent application Ser. No. 10/307,263 entitled “Magneto-Optical Bio-Discs and Systems Including Related Methods” filed Nov. 27, 2002. All of these applications are herein incorporated by reference in their entireties. They thus provide background and related disclosure as support hereof as if fully repeated herein.

More specifically, the present invention is directed to a method of extracting data from a bio-disc including the steps of controlling a bio-disc drive, an A/D card, and a storage device concurrently wherein the set of controlling is performed by one computer. In one implementation of this method, the extracting data uses a method of successive approximation. The method may further include using a set of assembly code in the step of controlling and, when desired, a set of parameters specific to the set of assembly code that enables omission of the instruction without loss of correctness to thereby allow the set of assembly code to omit an instruction that is absent from traditional compilers. In this particular embodiment, the instruction may be an underflow flag check instruction that is unnecessary because it would follow a subtraction operation wherein due to constraints on the inputs of the subtraction operation, an underflow occurrence is prohibited.

According to another aspect of this invention, there is provided a method of extracting data from a bio-disc. This method includes the steps of determining one or more locations on the bio-disc; positioning a head of a bio-disc drive to read the one or more locations; and sampling a signal generated by the head. In this method, the step of determining may include obliterating one or more ditches on the bio-disc, each of the ditches having a leading edge and a trailing edge; separating each of the ditches from each of the locations; and placing a template over the bio-disc. Each of the locations may be of a fixed rectangular size or of a fixed circular size. The separation between each of the ditches and each of the locations is fixed in one embodiment. In another, one of the locations is preceded by a pair of ditches separated by a fixed distance. Each of the ditches may be divided into a grid. In the grid embodiment, the grid preferably has a plurality of vertical sections corresponding to a number of bits in a binary address encoding of the ditch. Alternatively, the grid has a plurality of horizontal sections corresponding to a number of bit combinations in the binary address encoding of the ditch. In any of there grid implementations, each section of the grid including one of the horizontal sections and one of the vertical sections is burnt to indicate a binary one. Alternatively, each section of the grid comprising one of the horizontal sections and one of the vertical sections may be not burnt to indicate a binary zero. The template may advantageously include a set of markings that indicate radial distance.

In this second principal method, the step of positioning may further include the step of entering a radial distance, or alternatively entering a time length. In one specific implementation hereof, the step of positioning further includes (1) moving the head of the bio-disc drive forward at fixed intervals until the head encounters the leading edge of a first of the ditches, (2) moving the head of the bio-disc drive forward at fixed intervals when the head is over the first ditch, (3) moving the head of the bio-disc drive backwards at fixed intervals when the head leaves the ditch until the head encounters the trailing edge of the first ditch, (4) moving the head of the bio-disc drive forward at fixed intervals until the head encounters a first of the locations that is separated from the first ditch by a fixed distance, and (5) moving the head of the bio-disc drive forward at fixed intervals until the head encounters a next location.

Also in this embodiment, the step of sampling may include the further steps of entering a decrease in strength of the signal when the signal enters one of the ditches; entering an increase in strength of the signal when the signal leaves one of the ditches; entering a sample rate; and obtaining at the sample rate a value read by the head. In a similar manner, the step of determining may further include the step of entering a first number of sample regions to be sampled. This particular implementation, may include the further step of de-interleaving a sampled signal into a second number of files wherein the first number is equal to the second number and, when desired, storing the second number of files in a storage device. The step of storing may be performed concurrently with the step of sampling. In a particular implementation of this method, the step of determining may alternatively include the step of ordering a plurality of regions on the bio-disc. And more particularly, the step of ordering may include determining a specific region marker. The specific region marker may be a thickest region marker, a thickest region marker, or a thinnest region marker that may be implemented as a radial line. The step of ordering may further include determining an order for the plurality of regions relative to a known location on the bio-disc, and the step of determining may include detecting a marking on an outer region of the bio-disc. Here, the marking is part of an encoding scheme. And according to one aspect of this particular method, a radial position of the head remains stationary during the step of sampling.

According to another principal aspect of this invention, there is also provided another method of extracting data from a bio-disc. This method includes the steps of entering a desired centrifuge speed; and positioning a head of a bio-disc drive at a radial distance, the bio-disc drive directed to spin the bio-disc at the desired centrifuge speed when the head is positioned at the radial distance. This method may further include the steps of entering a duration and maintaining the head at the radial distance for the duration.

There is also provided a method of extracting data from a bio-disc comprising encoding a script that controls the behavior of a bio-disc data extraction system. In this embodiment, the script includes a sample rate. The script may also include a number of sample regions, a radial distance, a time length, a centrifuge speed, a centrifuge duration, or an incubation duration.

In any of the above methods, the instruction may also be an underflow flag check instruction associated with a subtraction operation wherein a first value involved in the subtraction operation is known to be larger than a second value involved in the subtraction operation wherein the second value is subtracted from the first value.

In accordance with another embodiment of this invention, there is provided a bio-disc data extraction system including a computer configured to control a bio-disc drive, an A/D card, and a storage device. This system may include use of a successive approximation method, and further include a set of assembly code wherein the set is used by the computer. According to one aspect of this embodiment, the set of assembly code omits an instruction wherein the instruction is not omitted by traditional compilers and wherein a set of parameters specific to the set of assembly code enables omission of the instruction without loss of correctness. In one particular instance, the instruction is an underflow flag check instruction that is unnecessary because it would follow a subtraction operation wherein due to constraints on the inputs of the subtraction operation an underflow cannot occur.

According to yet another embodiment of the present invention, there is provided a bio-disc data extraction system that includes (1) a locator configured to determine one or more locations on the bio-disc, (2) a positioning unit configured to position a head of a bio-disc drive to read the one or more locations, and (3) a sampling unit configured to sample a signal generated by the head. In this embodiment, the locator may advantageously include (i) one or more ditches obliterated on the bio-disc, each of the ditches having a leading edge and a trailing edge, (ii) a separation between each of the ditches and each of the locations, and (iii) a template configured to be placed over the bio-disc. Each of the locations may be of a fixed rectangular size or of a fixed circular size.

In one specific implementation, the separation between each of the ditches and each of the locations is fixed, or one of the locations is preceded by a pair of ditches separated by a fixed distance. Each of the ditches may alternatively be divided into a grid. In this case, the grid may have a plurality of vertical sections corresponding to a number of bits in a binary address encoding of the ditch. Alternatively, the grid has a plurality of horizontal sections corresponding to a number of bit combinations in the binary address encoding of the ditch. Also, each section of the grid may have one of the horizontal sections and one of the vertical sections left un-burnt to indicate a binary zero. Alternatively, each section of the grid may have one of the horizontal sections and one of the vertical sections burnt to indicate a binary one. In some of these embodiments, the template may have a set of markings that indicate radial distance. In any of these embodiments, the positioning unit may include an input unit configured to obtain a radial distance, or an input unit configured to obtain a time length. More specifically, the positioning unit that is configured to position the head in association with the system may further include control means to (1) move the head of the bio-disc drive forward at fixed intervals until the head encounters the leading edge of a first of the ditches, (2) move the head of the bio-disc drive forward at fixed intervals when the head is over the first ditch, (3) move the head of the bio-disc drive backwards at fixed intervals until the head leaves the ditch until the head encounters the trailing edge of the first ditch, (4) move the head of the bio-disc drive forward at fixed intervals until the head encounters a first of the locations that is separated from the first ditch by a fixed distance, and (5) move the head of the bio-disc drive forward at fixed intervals until the head encounters a next location. In addition, the sampling unit may specifically include an input unit configured to enter a decrease in strength of the signal when the signal enters one of the ditches; an input unit configured to enter an increase in strength of the signal when the signal leaves one of the ditches; an input unit configured to obtain a sample rate; and an obtainer configured to obtain at the sample rate a value read by the head. In one embodiment, the locator comprises an input unit configured to obtain a first number of sample regions to be sampled. The system may also include a de-interleaving unit configured to de-interleave a sampled signal into a second number of files. In one specific rendition, the first number is equal to the second number. The system may also have a storage device configured to store the second number of files, wherein storage of the second number of files is preferably performed concurrently with the sampling by the sampling unit. The locator may comprise an ordering unit configured to order a plurality of regions on the bio-disc. In this embodiment, the ordering unit may comprise a determiner configured to identify a region marker having a distinguishing feature. In one particular rendition, the distinguishing feature of the region marker is a predetermined thickness of the region marker, which may be a radial line. The ordering unit may also comprise a determiner configured to determine an order for the plurality of regions relative to a known location on the bio-disc, and the locator may comprise a detection unit configured to detect a marking on an outer region of the bio-disc. In some embodiments, the marking is part of an encoding scheme, and a radial position of the head remains stationary while the sampling unit samples the signal.

According to still yet another aspect of this invention, there is also provided a data extraction system for use with an optical analysis disc. This system includes a positioning unit configured to position a head of a disc drive at a radial distance; an input unit configured to obtain a desired centrifuge speed for the disc drive; and a controller to direct spin of the disc at the desired centrifuge speed when the head is positioned at the radial distance. This system may further include a second input unit configured to obtain a duration so that the head is maintained at the radial distance for the duration.

The present system further provides an optical analysis disc data extraction system comprising a script that controls the behavior of the disc data extraction system. In this embodiment, the script may include a sample rate, a number of sample regions, a radial distance, a time length, a centrifuge speed, a centrifuge duration, and/or an incubation duration.

In any of these extraction systems, the instruction may also be an underflow flag check instruction associated with a subtraction operation wherein a first value involved in the subtraction operation is known to be larger than a second value involved in the subtraction operation wherein the second value is subtracted from the first value.

In accordance with yet a further aspect of this invention, there is provided a computer program product that includes (1) a computer usable medium having a computer readable program code embodied therein configured to extract data from an optical analysis disc and (2) a computer readable code configured to cause a computer to control an optical analysis disc drive, an A/D card, and a storage device. In one embodiment, the computer readable program code implemented to extract the data from the optical analysis disc is a successive approximation program code. In another embodiment, the computer readable program code is derived from a set of assembly code. The computer readable code may be advantageously configured to cause a computer to concurrently control the optical analysis disc drive, the A/D card, and the storage device. In this specific implementation, the computer program product may further include a set of parameters specific to the set of assembly code, the set of assembly code omitting an instruction that enables omission of the instruction without loss of correctness. In this case, the instruction may be an underflow flag check instruction that is unnecessary because it would follow a subtraction operation wherein due to constraints on the inputs of the subtraction operation an underflow cannot occur.

According to still another aspect of the present invention, there is also provided a computer program product that includes (1) a computer usable medium having a computer readable program code embodied therein configured to extract data from a optical analysis disc; (2) a first computer readable code configured to cause a computer to determine one or more locations on the optical analysis disc; (3) a second computer readable code configured to cause the computer to position a head of a disc drive to read the one or more locations; and (4) a third computer readable code configured to cause the computer to sample a signal generated by the head. In one particular embodiment thereof, the first computer readable code, the second computer readable code, and the third computer readable code are integrated into a complete system code package that is loadable into a respective disc drive system to thereby enable the disc drive system to run the code package to analyze a sample associated with the optical analysis disc. The first computer readable code may include a fourth computer readable code configured to cause the computer to obtain a value generated from obliterating one or more ditches on the optical analysis disc, each of the ditches having a leading edge and a trailing edge; a fifth computer readable code configured to cause the computer to obtain a value generated from separating each of the ditches from each, of the locations; and a sixth computer readable code configured to cause the computer to obtain a value generated from a template configured to be placed over the optical analysis disc. In one product configuration, the fourth computer readable code, the fifth computer readable code, and the sixth computer readable code are integrated into a complete system code package that is loadable into a respective disc drive system to thereby enable the disc drive system to run the code package to analyze a sample associated with the optical analysis disc. In another product configuration, the first, second, third, fourth, fifth, and sixth computer readable codes are integrated into a complete system code package that is loadable into a respective disc drive system to thereby enable the disc drive system to run the code package to analyze a sample associated with the optical analysis disc. Each of the locations may be of a fixed rectangular size or of a fixed circular size. A separation between each of the ditches and each of the locations may be fixed. Also in one specific embodiment, one of the locations is preceded by a pair of ditches separated by a fixed distance. In another embodiment, each of the ditches is divided into a grid. In this case, grid has a plurality of vertical sections corresponding to a number of bits in a binary address encoding of the ditch. The grid may have a plurality of horizontal sections corresponding to a number of bit combinations in the binary address encoding of the ditch. Also, each section of the grid may have one of the horizontal sections and one of the vertical sections burnt to indicate a binary one. Alternatively, each section of the grid may have one of the horizontal sections and one of the vertical sections left un-burnt to indicate a binary zero. The template may have a set of markings wherein the set of markings indicate radial distance.

In one specific implementation of this computer program product, the second computer readable code comprises a fourth computer readable code configured to cause the computer to obtain a radial distance. As above, in this specific implementation, the first, second, third, and fourth computer readable codes may be integrated into a complete system code package that is loadable into a respective disc drive system to thereby enable the disc drive system to run the code package to analyze a sample associated with the optical analysis disc. Alternatively, the second computer readable code may comprise a fourth computer readable code configured to cause the computer to obtain a time length. Similarly, in this alternate case, the first, second, third, and fourth computer readable codes may be integrated into a complete system code package that is loadable into a respective disc drive system to thereby enable the disc drive system to run the code package to analyze a sample associated with the optical analysis disc.

Also, the second computer readable code may further alternatively comprise a fourth computer readable code configured to cause the computer to move the head of the disc drive forward at fixed intervals until the head encounters the leading edge of a first of the ditches; a fifth computer readable code configured to cause the computer to move the head of the disc drive forward at fixed intervals when the head is over the first ditch; a sixth computer readable code configured to cause the computer to move the head of the disc drive backwards at fixed intervals when the head leaves the ditch until the head encounters the trailing edge of the first ditch; a seventh computer readable code configured to cause the computer to move the head of the disc drive forward at fixed intervals until the head encounters a first of the locations that is separated from the first ditch by a fixed distance; and an eighth computer readable code configured to cause the computer to move the head of the disc drive forward at fixed intervals until the head encounters a next location. In this case, the computer program product may have the first, second, third, fourth, fifth, sixth, seventh, and eighth computer readable codes integrated into a complete system code package that is loadable into a respective disc drive system to thereby enable the disc drive system to run the code package to analyze a sample associated with the optical analysis disc.

In another implementation, the third computer readable code comprises a fourth computer readable code configured to cause the computer to obtain a sample rate; and a fifth computer readable code configured to cause the computer to obtain at the sample rate a value read by the head. Similarly with this implementation, the first, second, third, fourth, and fifth computer readable codes may be integrated into a complete system code package that is loadable into a respective disc drive system to thereby enable the disc drive system to run the code package to analyze a sample associated with the optical analysis disc.

In yet another implementation, first computer readable code comprises a fourth computer readable code configured to cause the computer to obtain a first number of sample regions to be sampled. Also here, the first, second, third, and fourth computer readable codes may be integrated into a complete system code package that is loadable into a respective disc drive system to thereby enable the disc drive system to run the code package to analyze a sample associated with the optical analysis disc. More specifically, this version of the computer program product may further comprise a fifth computer readable code configured to cause a computer to de-interleave a sampled signal into a second number of files. Here also, the first, second, third, fourth, and fifth computer readable codes are preferably integrated into a complete system code package that is loadable into a respective disc drive system to thereby enable the disc drive system to run the code package to analyze a sample associated with the optical analysis disc.

In one specific embodiment of this computer program product, the first number is equal to the second number. Another specific embodiment of this computer program product, may further include a sixth, computer readable code configured to cause the computer to store the second number of files in a storage device wherein storage of the second number of files is performed concurrently with sampling of the signal. In this product implementation, the first, second, third, fourth, fifth, and sixth computer readable codes may be preferably integrated into a complete system code package that is loadable into a respective disc drive system to thereby enable the disc drive system to run the code package to analyze a sample associated with the optical analysis disc.

In yet another embodiment of this computer program product, the first computer readable may alternatively comprise a fourth computer readable code configured to cause the computer to order a plurality of regions on the optical analysis disc. In this embodiment, the first, second, third, and fourth computer readable codes may be similarly integrated into a complete system code package that is loadable into a respective disc drive system to thereby enable the disc drive system to run the code package to analyze a sample associated with the optical analysis disc.

In still a further particular version of this embodiment, the fourth computer readable code may comprise a fifth computer readable code configured to cause the computer to determine a specific region marker. Here also, the first, second, third, fourth, and fifth computer readable codes may be integrated into a complete system code package that is loadable into a respective disc drive system to thereby enable the disc drive system to run the code package to analyze a sample associated with the optical analysis disc.

In yet still another alternate embodiment hereof, the first computer readable code may comprise a fourth computer readable code configured to cause the computer to detect a marking on an outer region of the optical analysis disc. In this case, the marking is preferably part of an encoding scheme. Similarly here, the first, second, third, and fourth computer readable codes may be preferably integrated into a complete system code package that is loadable into a respective disc drive system to thereby enable the disc drive system to run the code package to analyze a sample associated with the optical analysis disc. In any of these embodiments, a radial position of the head may preferably remain stationary while the signal is sampled.

And according to yet another principal aspect of this invention, there is also provided another computer program product that includes (1) a computer usable medium having computer readable program code embodied therein configured to extract data from an optical analysis disc; (2) computer readable code configured to cause a computer to obtain a desired centrifuge speed; and (3) computer readable code configured to cause a computer to position a head of a disc drive at a radial distance wherein the disc drive spins the optical disc at the desired centrifuge speed when the head is positioned at the radial distance. This computer program product may advantageously further comprise computer readable code configured to cause the computer to obtain a duration wherein the head is maintained at the radial distance for the duration.

According to still another principal aspect of this invention, there is provided yet another computer program product that includes (1) a computer usable medium having computer readable program code embodied therein configured to extract data from an optical analysis disc; and (2) computer readable code configured to cause a computer to execute a script that controls the behavior of an optical analysis disc data extraction system. In this embodiment of the present invention, the script may include a sample rate, a number of sample regions, a radial distance, a time length, a centrifuge speed, a centrifuge duration, and/or an incubation duration.

In any of the above summarized computer program products, the instruction may include an underflow flag check instruction associated with a subtraction operation wherein a first value involved in the subtraction operation is known to be larger than a second value involved in the subtraction operation wherein the second value is subtracted from the first value.

### BRIEF DESCRIPTION OF THE DRAWING

The above summarized inventions as well as other features, aspects, and advantages thereof will become better understood with regard to the following description, appended claims, and accompanying drawing figures where:

FIG. 1 is a pictorial representation of a bio-disc system;

FIG. 2 is an exploded perspective view of an example optical bio-disc;

FIG. 3 is a top plan view of the disc shown in FIG. 2;

FIG. 4 is block diagram showing the major components of the extraction system according to one aspect of the present invention;

FIG. 5A is a flow diagram of the process of centrifuging a bio-disc in accordance with one embodiment of the present invention;

FIG. 5B is a screen shot depicting the centrifuge control interface in the time-programmable mode according to one embodiment of the present invention;

FIG. 5C is a screen shot depicting the centrifuge control interface in the freehand mode according to one embodiment of the present invention;

FIG. 6 is a flow diagram of the process of finding desired sample regions on a bio-disc using ditches according to an embodiment of the present invention;

FIG. 7 is a graphical representation of a bio-disc with ditches each followed by a rectangular sampled area according to another aspect of the present invention;

FIG. 8 is a graphical representation of a bio-disc with ditches each followed by a circular sampled area according to a different embodiment of this aspect of the present invention;

FIG. 9 illustrates a ditch divided horizontally into 8 sections, and vertically into 3 sections according to one specific embodiment of this aspect of the present invention;

FIG. 10 illustrates a sampled area followed by a ditch to illustrate how individual cells within the sampled area can be catalogued according to an analysis method of the present invention;

FIG. 11 is a pictorial depiction of the measurement template for determining distance-based capture parameters;

FIG. 12 is a flow diagram of the process of capturing data from a bio-disc in accordance with one embodiment of the present invention;

FIG. 13 is a flow diagram of the process of performing a stationary capture in accordance with another embodiment of the present invention;

FIG. 14 is a schematic representation of a bio-disc from which data is being captured from four different regions in accordance with another aspect of the present invention;

FIG. 15 is a schematic representation of a bio-disc from which data is being captured from four different regions separated by radial lines in accordance with yet another aspect of the present invention;

FIG. 16 is a flow diagram of the process of capturing data for a user-defined number of capture regions on a bio-disc according to the present invention;

FIG. 17 is a flow diagram of the process of capturing data for a user-defined number of capture regions on a bio-disc using logical radial lines to separate regions in accordance with the present invention;

FIG. 18 is a screen shot showing the various options that can be input by the user in the capture control interface; and

FIG. 19 is a flow diagram of the process of capturing data from a bio-disc using a script in according to the present invention.

### DETAILED DESCRIPTION OF THE INVENTION

The invention is directed to methods and apparatus for extracting data from an optical bio-disc. In the following description, numerous specific details are set forth to provide a more thorough description of embodiments of the invention. In view of the present disclosure, however, it should be apparent to those skilled in the art, that the invention may be practiced without these specific details. In other instances, well known features have not been described in detail so as not to obscure the invention.

Bio-Disc System

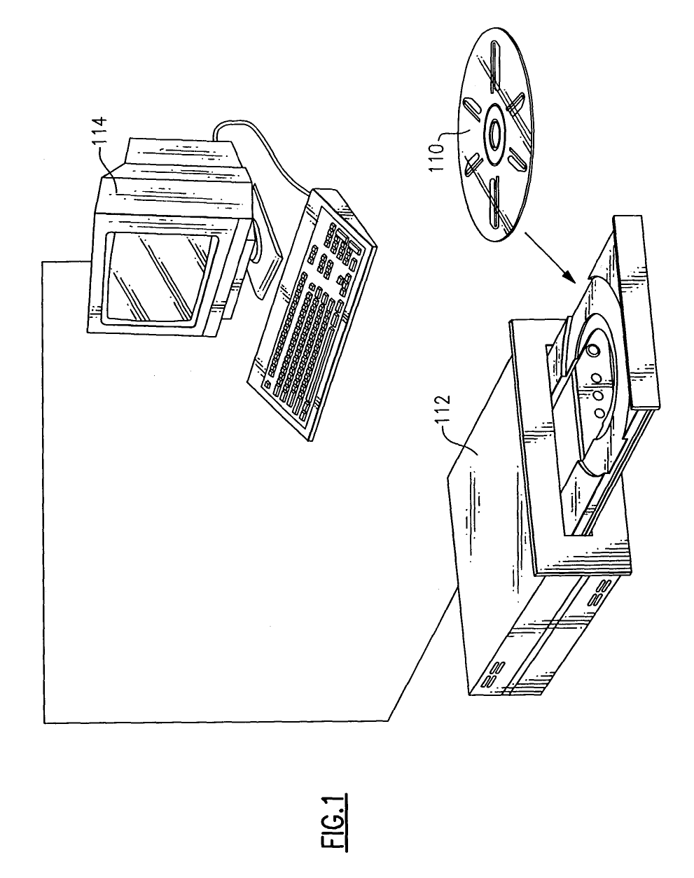

In accordance with one embodiment of the present invention, optical bio-drives are implemented as cost-efficient and effective alternatives for conducting cell counting and biological sample assays. An example optical bio-drive system configuration is shown in FIG. 1 in accordance with one embodiment of the present invention. Optical bio-disc 110, with fluidic channels housing biological samples is inserted into an optical disc drive 112. The optical features within optical disc drive 112 conduct biological assays on the samples housed within optical bio-disc 110. The optical mechanism of the optical disc drive 112 directs its laser beam at optical bio-disc 110 and uses a detector to detect reflected and/or scattered light. The detected light is converted to signal, which is converted to data that can be analyzed by computer 114. Monitor of display computer 114 displays the results of the assays.

Bio-Discs

In accordance with various embodiments of the present invention, a bio-disc is similar to a CD or DVD. In one embodiment, in addition to storing audio, visual and/or other data, a bio-disc may be used to diagnose certain ailments inside or outside of a doctor's office. Bio-discs may be utilized in home medical testing ranging from pregnancy tests to testing for cancer or the Ebola virus. In one embodiment, a test sample (e.g., urine or blood) is placed in a receptacle of the bio-disc and is tested by various means. In another embodiment, the fluid is forced past reactive regions in the disc. In yet another embodiment, the fluid and/or the regions are analyzed to determine the test results.

In one embodiment, to analyze the fluid or regions, a laser is directed towards the desired location. As the laser light hits the desired location, some or all of the light is absorbed, reflected, or passes through the sample after a light/matter interaction between the incident laser beam and the biological, chemical, or biochemical material being assayed. In some embodiments, bio-disc readers measure the amount of light reflected, and in other embodiments, bio-disc readers measure the amount of light that passes through the bio-disc. In one embodiment, this measurement produces a continuous signal that is sampled at a sample rate (i.e., the number of times the measured signal is sampled during a time period).

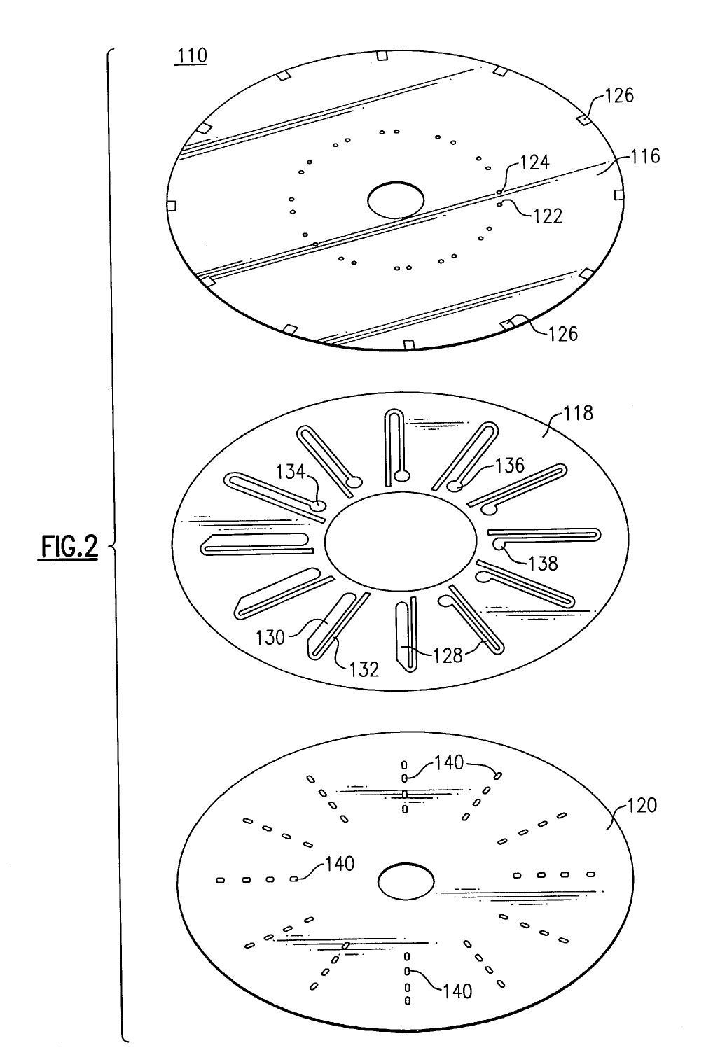

FIG. 2 shows an example optical bio-disc in accordance with one embodiment of the present invention. FIG. 2 is an exploded perspective view of the principal structural elements of the optical bio-disc 110. The principal structural elements include a cap portion 116, an adhesive member or channel layer 118, and a substrate 120. The cap portion 116 includes one or more inlet ports 122 and one or more vent ports 124.

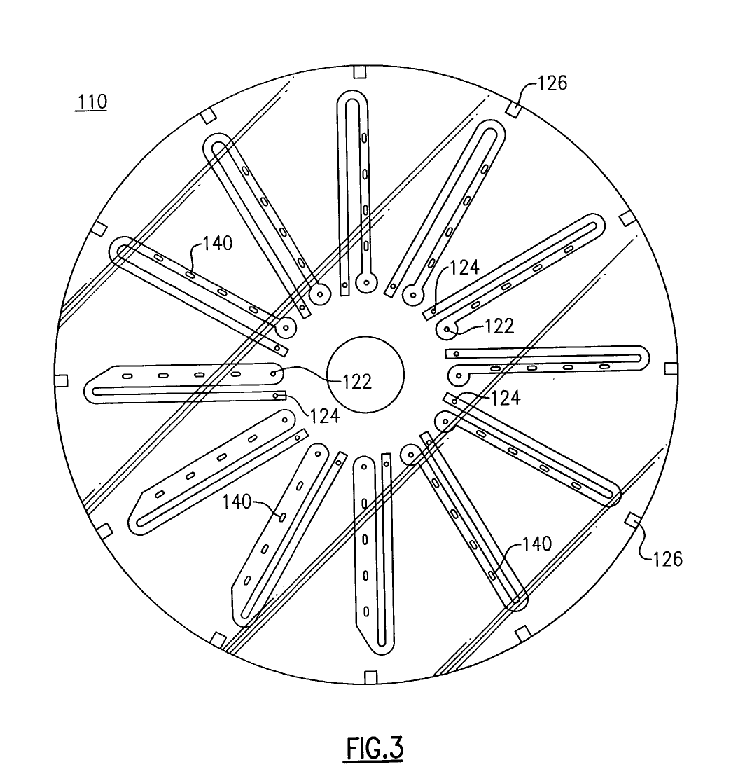

The second element shown in FIG. 2 is an adhesive member 118 having fluidic circuits 128 or U-channels formed therein. The fluidic circuits 128 are formed by stamping or cutting the membrane to remove the plastic film and form the shapes as indicated. Each of the fluidic circuits 128 includes a flow channel 130 and a return channel 132. Some of the fluidic circuits 128 illustrated in FIG. 3 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

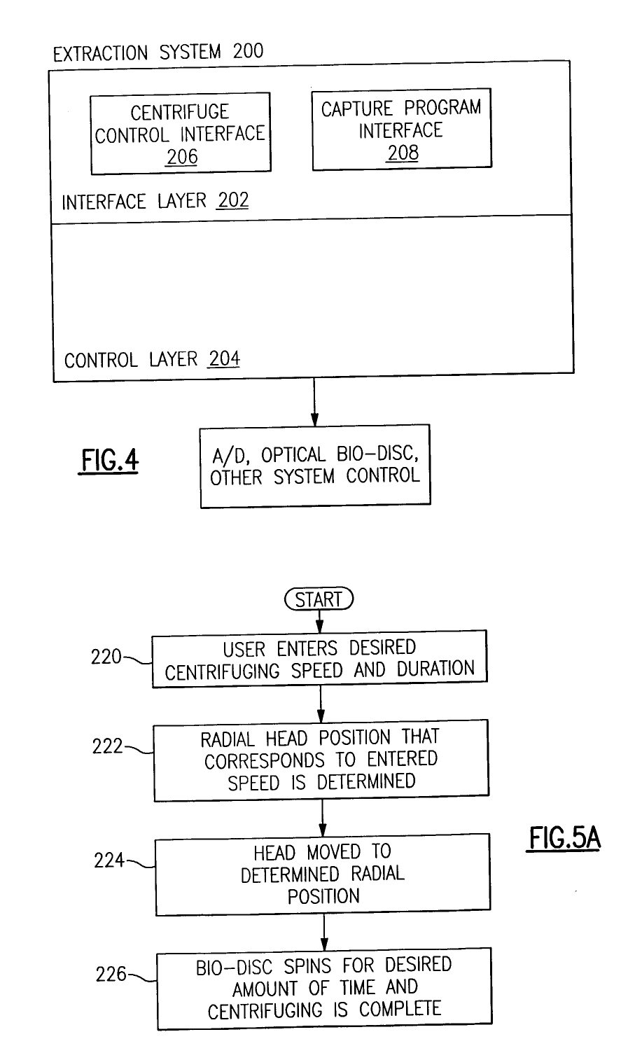

The third element illustrated in FIG. 2 is a substrate 120 including target or capture zones 140. The substrate 120 is preferably made of polycarbonate and has a reflective metal layer 142 deposited on the top thereof as also illustrated in FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum, gold, silver, nickel, and reflective metal alloys.

FIG. 3 is a top plan view of the optical bio-disc 110 illustrated in FIG. 2 with the reflective layer 142 on the cap portion 116 shown as transparent to reveal the fluidic circuits 128, the target zones 140, and trigger markings 126 situated within the disc.

During development and use of bio-discs, it is frequently useful to alter the sample rate to achieve the best test accuracy. Additionally, it is frequently useful to alter the number of regions being examined. In one embodiment, first generation software systems for extracting data from bio-discs as developed by the inventors hereof, had the sample rate and number of regions fixed.

First Generation Data Extraction Systems

In one embodiment, first generation data extraction systems developed by the inventors hereof were limited, due to computational demands, in their ability to execute disc operation control software, capture received signal data on an Analog-to-Digital (A/D) card, and store the captured data to a hard drive using only one computer. Because of early lower efficiencies in the compiled code written to perform each of these tasks, a single CPU was incapable of performing these tasks at the desired rate (e.g., capturing at 10 to 20 megacycles and storing data at 80 megabytes per second). Thus, the system shown in FIG. 1 in accordance with one embodiment of the present invention requires two computers and interconnecting hardware. In one embodiment, a first computer is used with an A/D card to perform the A/D capture as well as to store the captured information on a hard drive. A second computer was used to control the disc being monitored.

In various embodiments, this two-computer system required a high degree of manual control. For example, to capture a sample in one embodiment, a user would determine the location of the sample on a bio-disc, estimate the amount of time required for the head to be positioned over that location, initiate head movement using one computer, wait the estimated time period, and initiate capturing and storage using the second computer. Although embodiments using first generation data extraction systems are pioneering in nature, later developed embodiments of the present invention are more consistent and efficient.

Overview of Improved Extraction System

FIG. 4 describes the main components of the extraction system of the present invention. Extraction system 200 is a software system that comprises two main layers, interface layer 202 and control layer 204. Interface layer 202 allows user to control the various aspects of data extraction while control layer 204 controls the hardware that extracts data (e.g. A/D card, optical bio-drive mechanism) with the aid of support code. The interface layer comprises two main components, centrifuge control interface 206 and capture program interface 208. The control layer 204, on the other hand, contains software code such as A/D code needed to talk to an A/D card, Advanced Small Computer Systems Interface (SCSI) Programming Interface (ASPI) code needed to talk to a CD drive, or support routines such as de-interleaving data.

Control Layer and the Single Computer Extraction System

In one embodiment of the present invention, the process of capturing data from an optical bio-disc is controlled by a single computer. Control layer 204 in the extraction system is written in an assembly language. Using programming shortcuts from the assembly language programming art, but previously not applied to bio-disc extraction systems, one computer is capable of controlling the optical bio-drive, A/D card and storage drive simultaneously. The lower processing power required by control layer 204 of the present invention enables the use of a single computer extraction system.

An example of a shortcut is involved in a subtraction operation. High-level compilers generate code that performs the subtraction and then checks a flag to determine if underflow occurred. However, when it is known that underflow is not going to occur because of the restrictions on the input to the subtraction operation, the flag checking operation is unnecessary. In one embodiment, the code written in assembly does not perform this unnecessary flag checking operation. Shortcuts such as this are well-known to assembly code programmers, but they were not applied to prior art bio-disc data extraction systems.

Data Skipping

According to one embodiment of the present invention, it is possible to use a single computer extraction system using a method of data skipping such as successive approximation rather than sequential analysis to detect features on a disc. By not sequentially looking at all the sampling data that comes off the A/D card, but approximating the data based on prior results, the system can not only use a single computer, but also process the data in real time. Since the data is not stored before being processed, the user not only saves on storage costs, but can also obtain results in real time.

Centrifuge Control Interface

In the extraction system of the present invention, the head of the optical bio-drive is maintained at a constant radial distance to cause the bio-disc to act as a centrifuge at a desired speed. Because common disc-drive mechanisms speed up or slow down the rotational rate in accordance with the position of the head to keep a constant angular velocity, moving the head will cause the rotational rate to change. The extraction system takes advantage of this fact to control the rotational rate of the optical bio-disc.

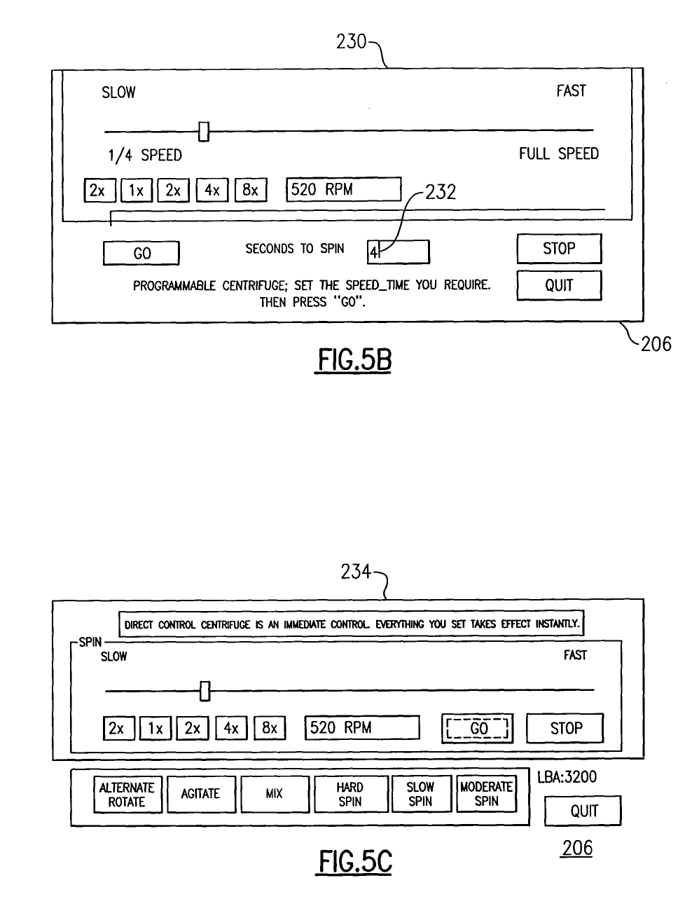

In one embodiment, the user inputs a desired centrifuge speed and the corresponding duration via centrifuge control interface 206, FIG. 4. Then control layer 204 directs the head of the optical bio-drive to automatically move to the corresponding radial distance for the desired duration to achieve the rotational rate with the aid of a CD drive software program such as Advanced SCSI Programming Interface (ASPI). FIG. 5A illustrates the process of centrifuging a bio-disc in accordance with one embodiment of the present invention. At block 220, a user enters a desired centrifuging speed (e.g., in rotations per minute, RPM) and duration. At block 222, the radial head position that corresponds to the entered speed is determined. At block 224, the head is moved to the determined radial position. At block 226, the bio-disc spins for the desired amount of time and centrifuging is complete.

FIG. 5B is a screen shot of the centrifuge control interface 206. The user can select the speed of the centrifuge by adjusting a slide bar, selecting a programmed speed of 1×, 2×, 4×, etc., or by typing in the RPM directly in speed selection interface 230. The user can then select the duration of the centrifuge by typing in the seconds to spin directly in duration selection interface 232.

A further option in centrifuge control interface 206 is the freehand centrifuge or direct control centrifuge. In this embodiment, the user can directly control the centrifuge process by speeding up or slowing down the rotation speed of the optical bio-drive. FIG. 5C is a screen shot of centrifuge control interface 206 in the direct control interface mode. The user can select from a set drive speed (1×, 2×, 4×, etc) or type in the RPM speed directly in speed selection interface 234. The duration of the centrifuge is controlled by the start and stop buttons and the RPM can be changed by the user at any time. Certain desired speeds may also be preset for specific assay requirements. These include, for example, “Agitate”, “Alternate Rotate”, “Mix”, “Hard Spin”, “Slow Spin”, or “Moderate Spin” as shown in FIG. 5C by way of example and not limitation. Thus, the drive motor may be advantageously controlled by the software aspects of the present invention to provide centrifugation. This may also include forward and reverse rotation of the drive to thereby perform the function of agitation or mixing of samples with reagents on the bio-disc in accordance with the differing requirements of the wide variety of assays that may be performed on the bio-disc.

A more detailed description on controlling optical bio-drive functions in the centrifuge operations is described in co-pending U.S. patent application titled, “Optical Analysis Disc and Related Drive Assembly for Performing Interactive Assembly for Performing Interactive Centrifugation”, Ser. No. 10/201,591, filed Jul. 22, 2002 which is hereby incorporated by reference.

Capture Program Interface

There are two main types of data capture operation that are available to the user via the capture program interface 208. They are (1) hardware triggered capture and (2) stationary capture. The user can select the type of capture most suited for the purpose of the particular assay conducted. Three main inputs are needed for both types of capture. These include sampling rate, disc rotation speed, and number of sample regions.

Hardware Triggered Capture Method

In this method of operation, the task of the capture program is to (1) determine the location of the sample regions on an optical bio-disc and (2) proceed with capturing data signal from detected light that has interacted with biological samples in those sample regions. There are several methods of locating the sample regions and proceeding with their capture. These several methods will now be discussed next.

According to one embodiment several ditches are obliterated on the disc. Moving clockwise (or the direction conventionally taken by a rotating disc), each ditch is made up of two edges (a leading edge and a trailing edge) separated by a 30 micron distance, in one preferred implementation of this embodiment. The area of interest or sampled region is, according to one embodiment, a rectangular region. This region may be of size 30×30 microns, and is placed behind the trailing edge of each ditch. There is a pair of ditches in front of one of the sampled regions. This triggers the primary trigger or spot number 1. The distance between the ditches of the primary trigger is a fixed distance, for example, 75 microns. The aim is to find the exact position of a ditch and hence the exact locations of the sampled areas.

In operation, the 30 micron wide ditches and the 30×30 micron sampled areas are placed peripherally and the laser is made to strike the disc at intervals of 20 microns. This interval ensures that the laser will strike the disc at the location of a ditch. A reduction in the signal strength triggers the computer extraction system that the laser has encountered a ditch. Once this happens, the laser is made to move forward at intervals of 2 microns until either the exact trailing edge of the ditch is encountered or the laser leaves the ditch. An increase in the signal strength triggers the computer extraction system that the laser has left the ditch. At this point, the laser is made to move backwards at intervals of half microns until the exact trailing edge of the ditch is encountered. This gives the exact location of a ditch. Once this process is performed, the laser is directed to move 75 microns forward. If the signal strength reduces, then the extraction system knows that the laser has encountered another ditch, and hence the primary trigger spot. Since the distance behind each ditch from where the sample area begins, and the duration of the sample area are fixed and known distances, once the system locates the exact position of a ditch, finding the location of all the sample areas is elementary.

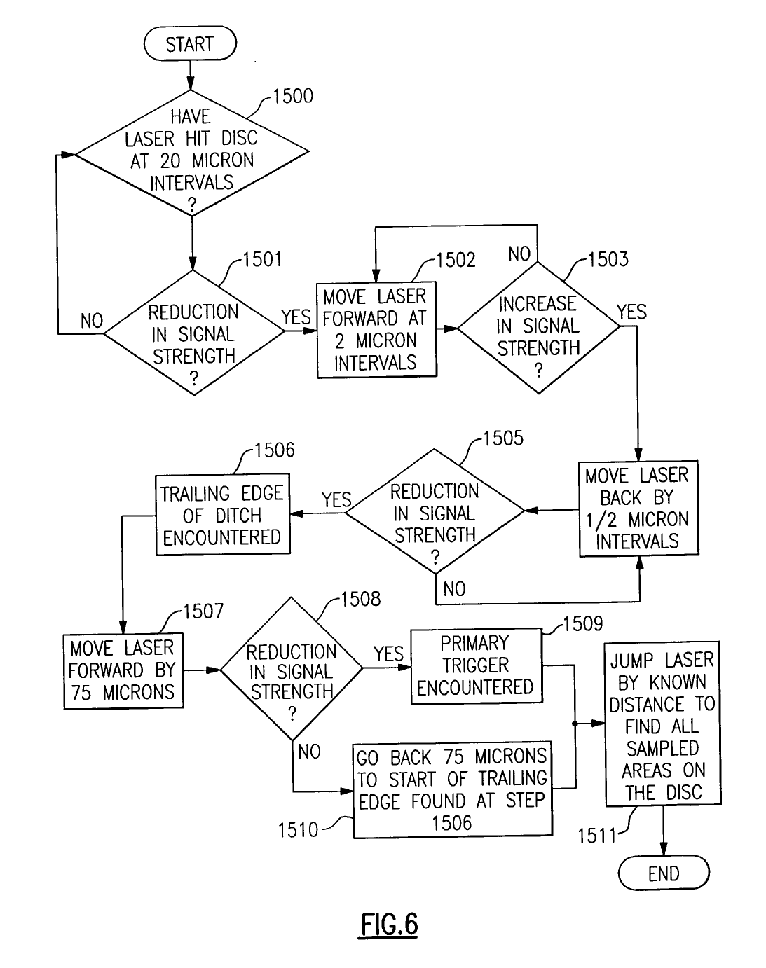

FIG. 6 is a flowchart that graphically shows the operation explained above. At step 1500 the laser is made to hit the disc at 20 micron intervals. At step 1501, a check is made to see if there is a reduction in signal strength. If there is no reduction (the “no” branch), then continue to hit the laser at 20 micron interval. If there is a reduction in signal strength (the “yes” branch), then a ditch is encountered, and at step 1502 the laser is made to move forward at intervals of 2 microns. At step 1503, a check is made to see if there is an increase in signal strength. If there is no increase (the “no” branch), then continue to move the laser forward in 2 micron intervals. If there is an increase in the signal strength (the “yes” branch), then the system knows that the laser has left the ditch, and at step 1504, the laser is made to move backwards in half micron intervals. At step 1505, another check is made to see if there is a reduction in signal strength. If there is no reduction (the “no” branch), then continue to move the laser back in half micron intervals. If there is a reduction in signal strength (the “yes” branch), then the system knows at step 1506 that it has found the trailing edge of the ditch it was in at step 1502. Next, at step 1507, the system moves the laser forward by a distance of 75 microns. At step 1508, another check is made to see if there is a reduction in signal strength. If there is (the “yes” branch), then the system knows that it has encountered the primary trigger mark (or spot number 1). If, on the other hand, there is no reduction in signal strength (the “no” branch), then the system moves the laser back by 75 microns to the start of the trailing edge found at step 1506. Once a ditch is found (either the primary or a secondary trigger), at step 1511 the laser is directed to jump known distances to find all the sampled areas on the disc.

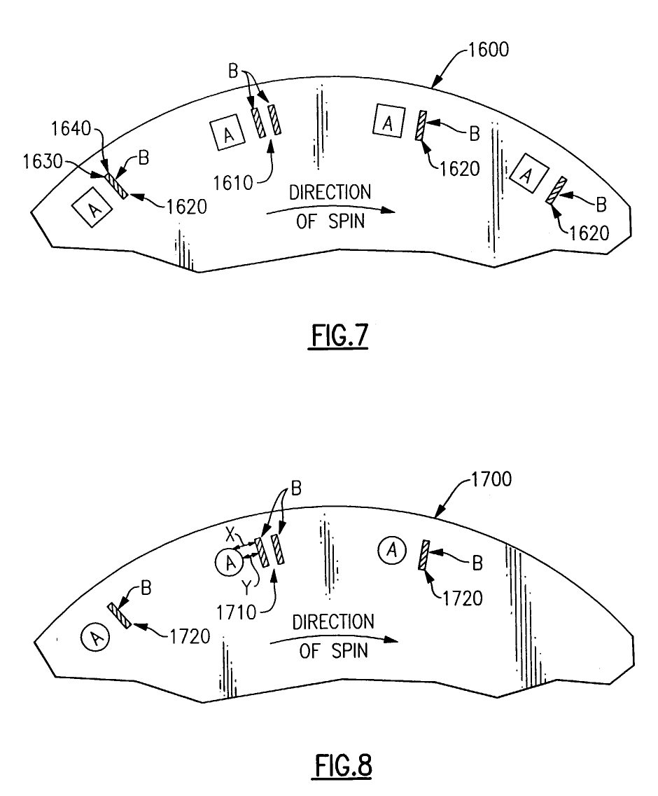

FIG. 7 is a graphical representation of a disc 1600 with ditches B each followed by a sampled area A at a fixed and known distance from B. The sizes of each sampled area and ditch are also fixed and known. Ditch 1610 is a primary trigger, and ditches 1620 are secondary triggers. Reference numerals 1630 and 1640 illustrate the trailing and leading edges of a ditch.

The area of interest or sampled region is, according to one embodiment, a circular region. A circular sampled area may be preferred over a rectangular sampled area because if the sampled chemistry is fluidic, then a circular area holds the chemistry better in place than a rectangular area once the disc starts spinning. But, since the circumferential distance of a circular object is different at different radial distances from the center, the laser, in operation, is directed to skip different distances depending on where on the disc it is tracking the sampled areas. But once the system knows the fixed distance between the sampled area and the ditch at that peripheral arc where the laser hits the disc, the operation of finding all sampled areas on the disc is similar to the steps explained in FIG. 6 above.

These differences in distance are illustrated in FIG. 8. Disc 1700 has ditches B similar to the ones discussed in FIGS. 6 and 7 above followed by a sampled area A at a fixed and known distance from B. The sampled area A, unlike the sampled areas discussed in FIGS. 6 and 7, is a circular sampled area and hence distance X is different from distance Y. But, since the sizes of each sampled area and ditch are fixed and known, and even though the distance between a ditch and a sampled area is different depending on where the laser is hitting the disc, calculating these different distances like X and Y is elementary once the user has fine tuned the laser to hit the disc at a known peripheral location. Similar to FIG. 7, ditch 1710 is the primary trigger, and ditches 1720 are secondary triggers. Reference numerals 1730 and 1740 illustrate the trailing and leading edges of a ditch.

Binary Address Encoding

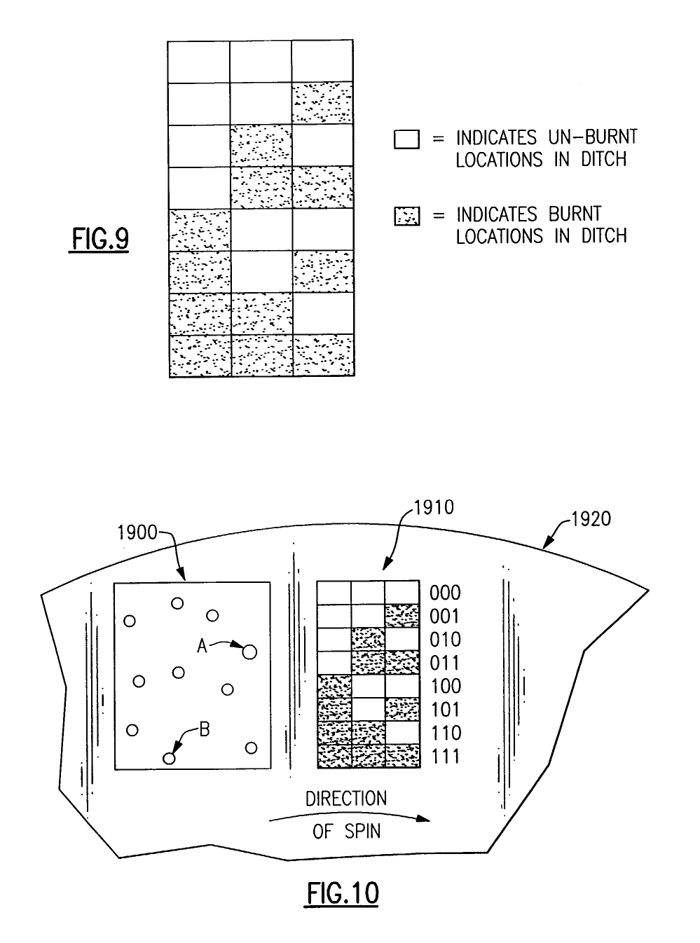

In one embodiment, the ditches have binary encoding obliterated inside them. One way of obliterating a binary code inside a ditch is to divide the ditch horizontally into sections. If the ditch is divided into 8 horizontal sections, then each one is binary encoded from 000 through 111. Similarly, if the ditch is divided into 16 horizontal sections, then each one is binary encoded from 0000 through 1111. In the case of 8 horizontal sections per ditch, the ditch is further divided vertically into 3 sections so that each horizontal row has a location for the 3 binary codes needed to represent that horizontal section. Similarly, in the case of 16 horizontal sections per ditch, the ditch is further divided vertically into 4 sections so that each horizontal row has a location for the 4 binary codes needed to represent that horizontal section. In one embodiment, zeros are burnt onto the disc, while ones are left un-burnt. FIG. 9 illustrates a ditch 1800 divided horizontally into 8 sections, and vertically into 3 sections. The horizontal sections are numbered 000 through 111. FIG. 9 also shows the burnt locations within the ditch to indicate ones and un-burnt locations within the ditch to indicate zeros.

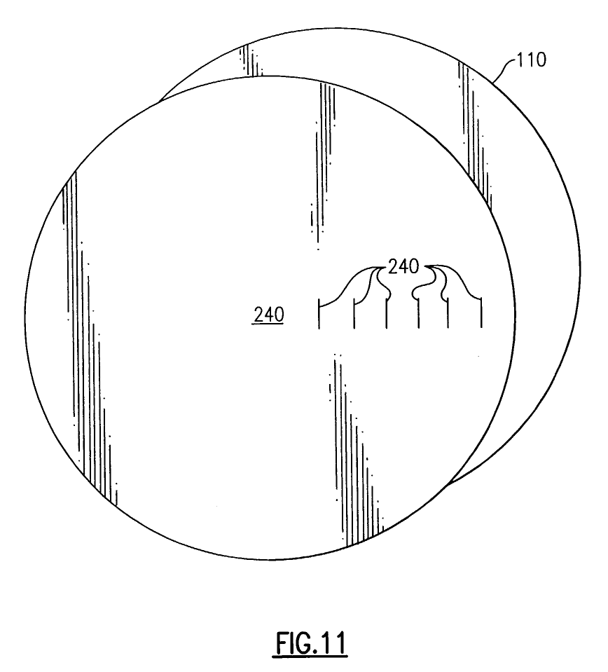

In operation, cells within a chemistry placed in the sampled area following a ditch, can now be catalogued based on the binary address encoding of the ditch. FIG. 10 illustrates a sampled area 1900 followed by a ditch 1910 on disc 1920. The ditch is divided into eight horizontal sections and three vertical sections, and are numbered from 000 through 111. Cells (indicated by the small circular areas in the figure) within the sampled area are catalogued based on their location derived from the binary addressing system of the ditch. For example, cell A may be catalogued as A010, where 010 indicates the horizontal row of ditch A where cell A may be found. Similarly, cell B may be catalogued as A111, where 111 indicates the horizontal row of ditch A where cell B may be found.

Sample Regions

An optical disc has numerous sample regions. As can be seen from the example optical bio-disc in FIGS. 2 and 3, there are numerous elements on the optical bio-disc and selecting the appropriate capture area on the disc is an important part of the function of the extraction system of the present invention.

Numerous bio-disc embodiments can be used in conjunction with the present invention. Other various disc embodiments are more fully described in co-pending U.S. patent application entitled “Multi-purpose Optical Analysis Optical Bio-disc for Conducting Assays and Various Reporting Agents for Use Therewith,” Ser. No. 10/194,396, filed on Jul. 12, 2002.

Since the user has the option of entering the location information into the capture interface, one embodiment of the present invention provides a template to assist in determining the location of one or more desired sample regions. The see-though template is placed over a bio-disc and markers on the template enable a user to determine a sample region's maximum and minimum radial distances on the bio-disc. After measuring the maximum and minimum radial distances, the user can enter the measured distances as input to the capture program interface. The program then calculates the correct moment to begin data capture using the input distances. In another embodiment, the control layer causes the head to move directly to the minimum radial distance entered by a user without first reading any other tracks.

FIG. 11 illustrates a measurement template in accordance with one embodiment of the present invention. The template 240 is made of a clear material. Markings 242 on the template 240 indicate radial distance. As shown, template 240 is to be placed over optical bio-disc 110. In one embodiment, the markings measure the distance in millimeters. In another embodiment, the markings measure the distance in inches. In other embodiments, other units of measurement are used.

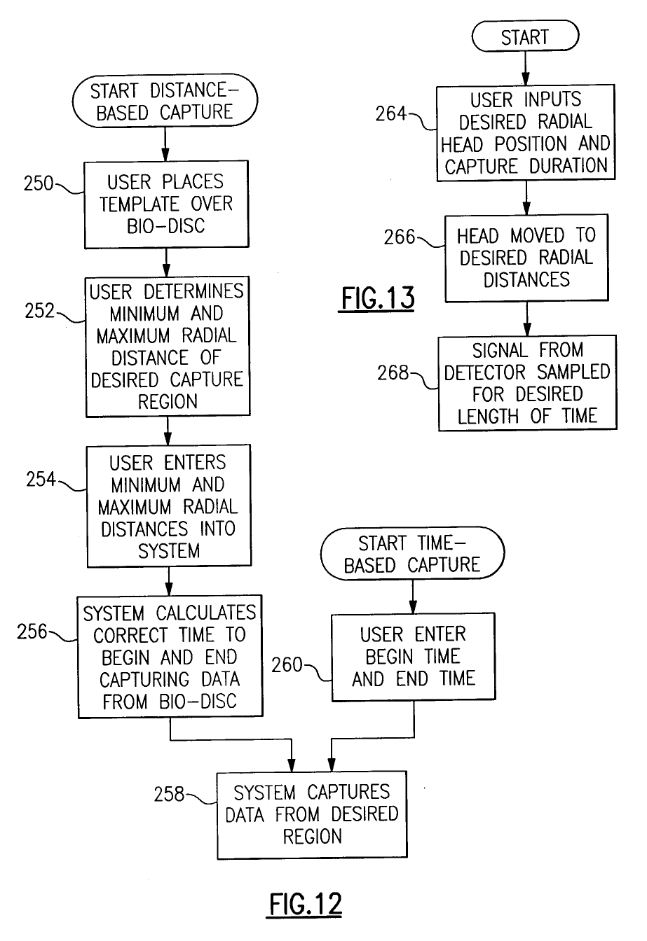

FIG. 12 illustrates the process of capturing data from a bio-disc in accordance with one embodiment of the present invention. At block 250, a user places a template over a bio-disc. At block 252, the user determines the minimum and maximum radial distance of the desired capture region. At block 254, the user enters the minimum and maximum radial distances into the system. At block 256, the system calculates the correct time to begin and end capturing data from the bio-disc. At block 258, the system captures data from the desired region.

FIG. 12 also shows another main method of hardware-triggered capture available to the user, time-based capture. In this method, the user enters a beginning and end time in the capture program interface 208 rather than a distance to control when the data capture begins. Block 260 of FIG. 12 shows this process.

Stationary Capture

In another embodiment, data is collected from the bio-disc while the head is maintained at a constant radial distance. As items in the biological sample (e.g., red blood cells) flow past the head position, the signal generated is altered. Thus, data is collected about changes in a region with respect to time. FIG. 13 illustrates the process of performing a stationary capture in accordance with one embodiment of the present invention. At block 264, a user inputs the desired radial head position and the capture duration. At block 266, the head is moved to the desired radial distance. At block 268, the signal from the detector is sampled for the desired length of time. In one embodiment, the user can select to have the head positioned at one of eight pre-defined spots when the extraction system is reading an eight-sample area optical bio-disc.

Capture Parameters

Regardless of the type of capture method selected, the extraction system of the present invention allows the user to specify the drive speed, sampling rate, and number of sample areas. In one embodiment, the drive speed of 1×, 2×, 4×, or 8× can be selected. The speed dictates how fast the optical bio-drive is to be spun while data capture is taking place. As would be readily apparent to one of skill in the drive art working with assay developers, additional drive speeds as desired may be easily provided.

Control layer 204, FIG. 4, is configured to sample at the rate input by the user without the need of reprogramming. In prior art systems, the sample rate was defined in the computer programming code. Thus, to change the sample rate, a programmer had to change the code. In this embodiment, the sample rate is treated in the code as a variable that is alterable by the user. In one embodiment, the sampling rate can range from 0.2 KHz to 40 MHz.

In addition to a user-specified sampling rate input, the extraction system of the present invention also allows users to specify the number of sample regions from which data are to be captured. The software automatically de-interleaves the captured data into the correct number of data files (i.e., each of the sample areas corresponds to a separate data file that is written to a storage device as the data is captured).

To accommodate the functionality of allowing the user to choose sample areas, optical bio-disc embodiments hereof as employed in conjunction with the extraction methods of present inventions, have capture areas that are designated by markings on the outer rim of the bio-discs. In one embodiment, the markings are made by placing an opaque substance (e.g., silk screen indicia) on the outer rim. As the disc rotates, a first detector determines when the markings are present. When the markings are present, a second detector collects light that has interacted with the corresponding sample area on the bio-disc. In one embodiment, the markings are also used to determine an ordering for the sample areas. In an example embodiment, the longest sequence of markings (e.g., the longest indica) is the first sample area in the ordering. In other embodiments, the markings are part of an encoding scheme. In one embodiment, the markings encode the beginning of a sample area. In another embodiment, the markings encode the end of a sample area. In yet another embodiment, markings encode the size of the sample area. In still another embodiment, markings encode the sample area's position in the ordering of sample areas.

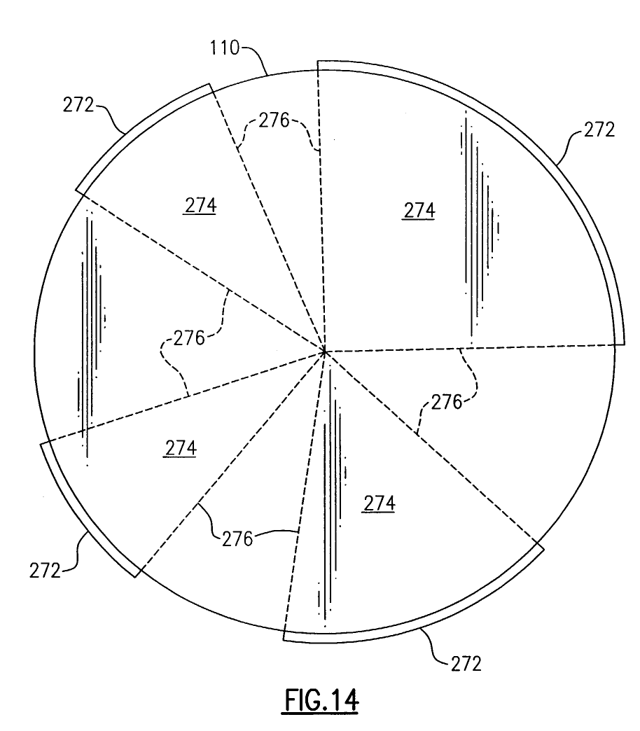

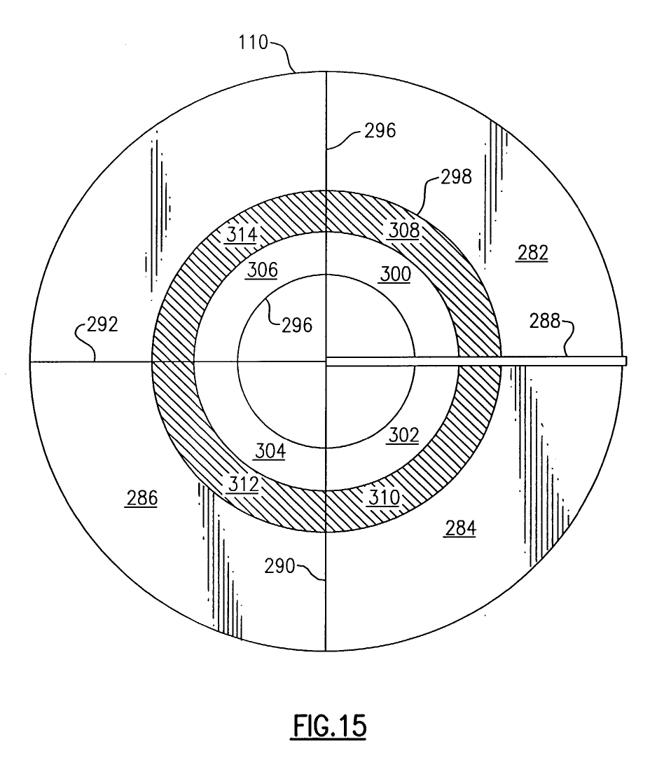

FIG. 14 illustrates an optical bio-disc from which data is being captured from four different regions in accordance with one embodiment of the present invention. The bio-disc 110 has marking 272 on its outer rim. The markings indicate that the sample regions 274 are located within the area bound by imaginary lines 276 that run from the ends of the markings to the center of the bio-disc. The sample regions can be further bound by defining maximum and minimum radial distances for the sample region.

In one embodiment, capture regions are separated by radial lines. FIG. 15 illustrates a bio-disc from which data is being captured from four different regions separated by radial lines in accordance with one embodiment of the present invention. The bio-disc 110 is divided into four regions 280, 282, 284 and 286 by radial lines 288, 290, 292 and 294. The sample areas are further defined by the minimum radial distance 296 of the sample areas and the maximum radial distance 298 of the sample areas. The number of sample areas (one or more) is determined by a user. The user can also set the minimum (296) and maximum (298) radial distances so that the entire disc is in the sample area.

Once the head is positioned between the minimum and maximum radial distances and the bio-disc rotates in the drive, tracks of each sample rotate past the head and are sampled. The two tracks on the example disc in FIG. 15 are distinguished by a circular white band and a circular shaded band. This is a simplification for purposes of illustration. As would be understood by one of skill in the optical disc art, actual physical tracks on a disc spiral out and are much smaller being on the order of 1.5 microns to sub-micron pitch in current DVD standards. Now back to the example in FIG. 15. The tracks may be read in the following order: track portion 300, track portion 302, track portion 304, track portion 306, track portion 308, track portion 310, track portion 312, and track portion 314. Thus, the data for each sample region is interleaved with data for other sample areas. The system de-interleaves the data into separate files. Thus, data from track portions 300 and 308 are stored in the same file. Similarly, track portions 302 and 310; 304 and 312; and 306 and 314 are stored together in three separate files. In one embodiment, each track for a sample region is stored as a new line in the storage file. In other embodiments, other storage schemes are used.

In one embodiment, the radial lines exist physically on the bio-disc. In an example embodiment, black adhesive strips or silk screened indicia are used to form the lines. The regions are numbered (ordered) starting at one of the radial lines and numbering either clockwise or counter-clockwise. In one embodiment, the thickest radial line is used to initiate numbering. For example in FIG. 15, radial line 288 is the thickest line and would indicate the first sample region. In another embodiment, the thinnest radial line is used to initiate numbering. In yet another embodiment, a line with a specific color is used to initiate numbering. In still another embodiment, a line with a specific pattern along an edge is used to initiate numbering.

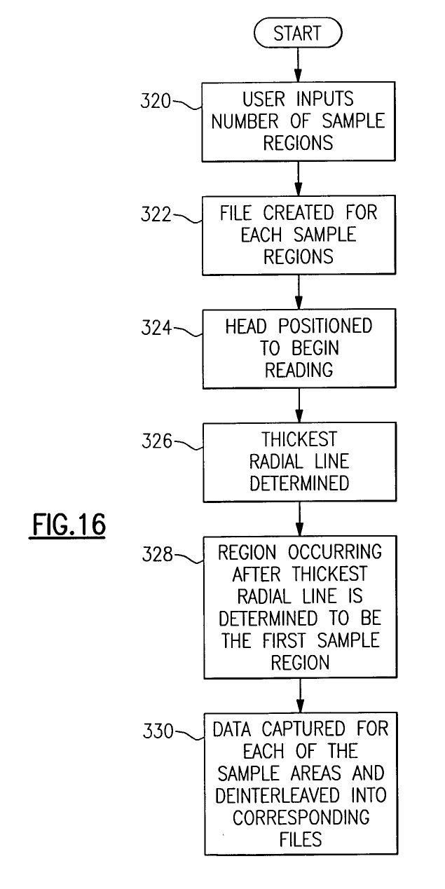

FIG. 16 illustrates the process of capturing data for a user-defined number of capture regions on a bio-disc in accordance with one embodiment of the present invention. At block 320, a user inputs the number of sample regions. At block 322, a file is created for each sample region. At block 324, the head is positioned to begin reading. At block 326, the thickest radial line is determined. At block 328, the region located after the thickest radial line is determined to be the first sample region. At block 330, data is captured for each of the sample areas and de-interleaved into the corresponding files.

In yet another embodiment, the radial lines are logical. In one embodiment, the location of the logical lines is determined relative to an identifiable sequence on the bio-disc. In another embodiment, a known region of the bio-disc encodes the location and number of sample regions.

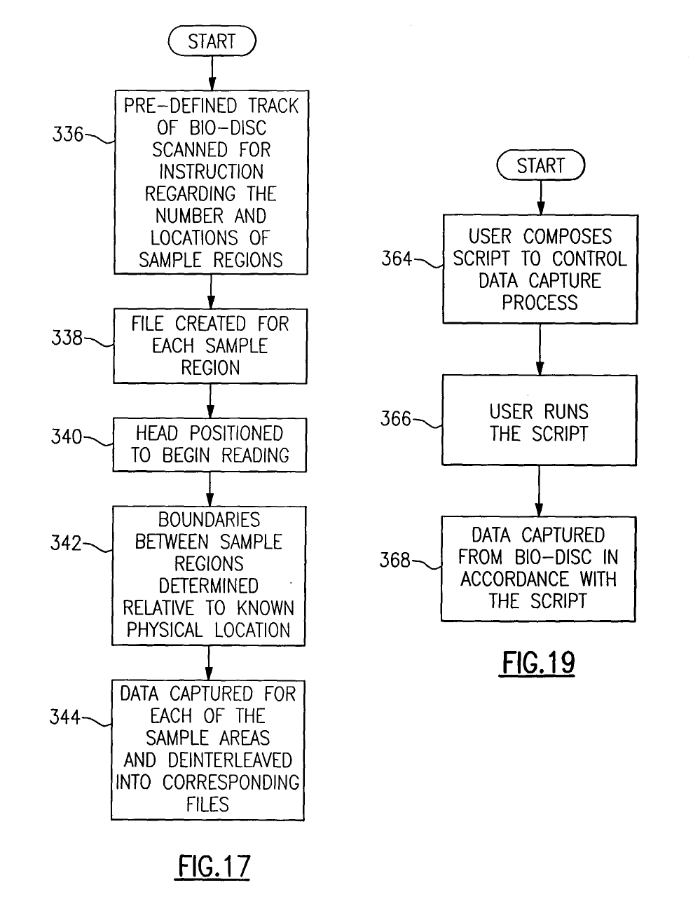

FIG. 17 illustrates the process of capturing data for a user-defined number of capture regions on a bio-disc using logical radial lines to separate regions in accordance with one embodiment of the present invention. At block 336, a pre-defined track of the bio-disc is scanned for instruction regarding the number and locations of sample regions. At block 338, a file is created for each sample region. At block 340, the head is positioned to begin reading. At block 342, the boundaries between sample regions are determined relative to a known physical location (e.g., the end of the instructions on the pre-defined track). At block 344, data is captured for each of the sample areas and de-interleaved into the corresponding files.

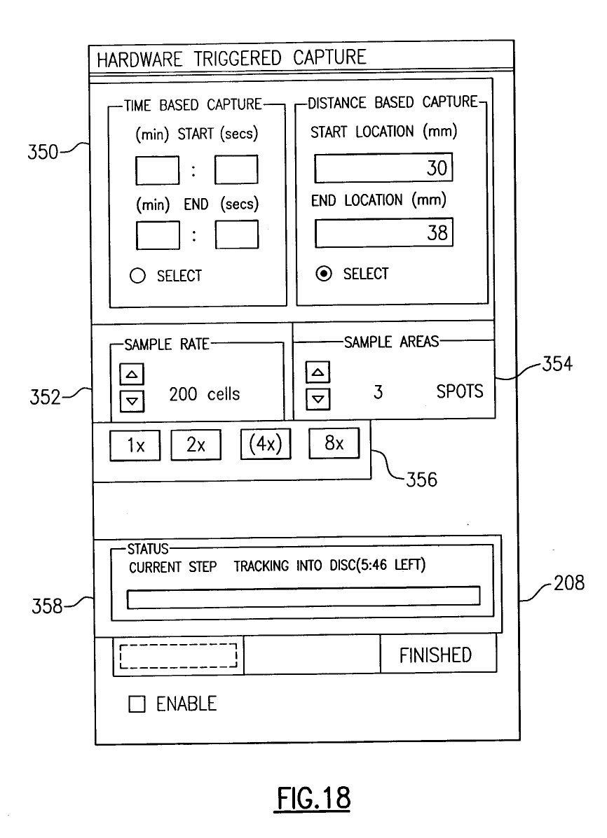

FIG. 18 shows a screen shot of capture control interface 208 in the hardware triggered capture embodiment. The user can choose between time-based capture and distance based capture in capture selection interface 350. Then the user can input the sampling rate in sample rate interface 352, and the number of sample areas in sample area selection interface 354. The user can also select the drive speed in drive speed selection interface 356. The status bar 358 displays the progress of the capture.

Scripting Control

In one embodiment, the behavior of the analysis system can be controlled using a scripting language. For example, the system can be scripted to centrifuge at a desired speed for a desired amount of time, incubate for another amount of time, and capture data from a desired region. FIG. 19 illustrates the process of capturing data from a bio-disc using a script in accordance with one embodiment of the present invention. At block 364, a user composes a script to control the data capture process. The script may specify one or more sample rates. These include, for example: the number and location of sample regions; whether, when, and how long to centrifuge; whether, when, and how long to pause for incubation; whether the head should remain stationary during the capture; and other standard control operations for an optical bio-drive. At block 366, the user runs the script. At block 368, data is captured from the bio-disc in accordance with the script.

Captured Data Output

Once the data signal is captured, the extraction system of the present invention allows user to select different format of output. The output can be a CSV (Comma Separated Value) file detailing the value detected at each sampling point, a BMP file depicting the intensity of light detected from the sample area, or other desired outputs. Furthermore, the user can choose to have a post-process performed on the output files. The post-process can analyze the data in the files for cell counting and recognition. A more detailed description of the post-process analysis that can be performed on the data files is disclosed in co-pending U.S. patent application entitled “Method and Apparatus for Differential Cell Counts and Related Software for Performing Same,” Ser. No. 10/241,512, filed Sep. 12, 2002 which is herein incorporated by reference.

Concluding Statements

Thus, methods and apparatus for extracting data from optical bio-discs have been described in conjunction with one or more specific embodiments. And, while this invention has been described in detail with reference to certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the literal meaning and range of equivalency of the claims are to be considered within their scope.

1. A method of extracting data from a bio-disc comprising controlling a bio-disc drive, an A/D card, and a storage device concurrently wherein said set of controlling is performed by one computer.

2. The method of claim 1 wherein said extracting data uses a method of successive approximation.

3. The method of claim 1 further comprising using a set of assembly code in said step of controlling.

4. The method of claim 3 further including a set of parameters specific to said set of assembly code that enables omission of said instruction without loss of correctness to thereby allow said set of assembly code to omit an instruction that is absent from traditional compilers.

5. The method of claim 4 wherein said instruction is an underflow flag check instruction that is unnecessary because it would follow a subtraction operation wherein due to constraints on the inputs of said subtraction operation, an underflow occurrence is prohibited.

6. A method of extracting data from a bio-disc, said method comprising the steps of:
determining one or more locations on said bio-disc;
positioning a head of a bio-disc drive to read said one or more locations; and
sampling a signal generated by said head.

7. The method of claim 6 wherein said step of determining further comprises:
obliterating one or more ditches on said bio-disc, each of said ditches having a leading edge and a trailing edge;
separating each of said ditches from each of said locations; and
placing a template over said bio-disc.

8. The method of claim 7 wherein each of said locations is of a fixed rectangular size.

9. The method of claim 7 wherein each of said locations is of a fixed circular size.

10. The method of claim 7 wherein separation between each of said ditches and each of said locations is fixed.

11. The method of claim 7 wherein one of said locations is preceded by a pair of ditches separated by a fixed distance.

12. The method of claim 7 wherein each of said ditches is divided into a grid.

13. The method of claim 12 wherein said grid has a plurality of vertical sections corresponding to a number of bits in a binary address encoding of said ditch.

14. The method of claim 12 wherein said grid has a plurality of horizontal sections corresponding to a number of bit combinations in said binary address encoding of said ditch.

15. The method of claim 12 wherein each section of said grid comprising one of said horizontal sections and one of said vertical sections is burnt to indicate a binary one.

16. The method of claim 12 wherein each section of said grid comprising one of said horizontal sections and one of said vertical sections is not burnt to indicate a binary zero.

17. The method of claim 7 wherein said template has a set of markings that indicate radial distance.

18. The method of claim 6 wherein said step of positioning further comprises the step of entering a radial distance.

19. The method of claim 6 wherein said step of positioning further comprises the step of entering a time length.

20. The method of claim 6 wherein said step of positioning further comprises:
moving said head of said bio-disc drive forward at fixed intervals until said head encounters said leading edge of a first of said ditches;
moving said head of said bio-disc drive forward at fixed intervals when said head is over said first ditch;
moving said head of said bio-disc drive backwards at fixed intervals when said head leaves said ditch until said head encounters said trailing edge of said first ditch;
moving said head of said bio-disc drive forward at fixed intervals until said head encounters a first of said locations that is separated from said first ditch by a fixed distance; and
moving said head of said bio-disc drive forward at fixed intervals until said head encounters a next location.

21. The method of claim 6 wherein said step of sampling comprises the further steps of:
entering a decrease in strength of said signal when said signal enters one of said ditches;
entering an increase in strength of said signal when said signal leaves one of said ditches;
entering a sample rate; and
obtaining at said sample rate a value read by said head.

22. The method of claim 6 wherein said step of determining further comprises the step of entering a first number of sample regions to be sampled.

23. The method of claim 22 further comprising the step of de-interleaving a sampled signal into a second number of files wherein said first number is equal to said second number.

24. The method of claim 23 further comprising storing said second number of files in a storage device, said step of storing being performed concurrently with said step of sampling.

25. The method of claim 6 wherein said step of determining further comprises the step of ordering a plurality of regions on said bio-disc.

26. The method of claim 25 wherein said step of ordering comprises determining a specific region marker.

27. The method of claim 26 wherein said specific region marker is a radial line.

28. The method of claim 26 wherein specific region marker is a thinnest region marker.

29. The method of claim 28 wherein said thinnest region marker is a radial line.

30. The method of claim 25 wherein said step of ordering comprises determining an order for said plurality of regions relative to a known location on said bio-disc.

31. The method of claim 6 wherein said step of determining further comprises detecting a marking on an outer region of said bio-disc.

32. The method of claim 31 wherein said marking is part of an encoding scheme.

33. The method of claim 6 wherein a radial position of said head remains stationary during said step of sampling.

34. A method of extracting data from a bio-disc, said method comprising the steps of:
entering a desired centrifuge speed; and
positioning a head of a bio-disc drive at a radial distance, said bio-disc drive directed to spin said bio-disc at said desired centrifuge speed when said head is positioned at said radial distance.

35. The method of claim 34 further comprising the steps of entering a duration and maintaining said head at said radial distance for said duration.

36. A method of extracting data from a bio-disc comprising encoding a script that controls the behavior of a bio-disc data extraction system.

37. The method of claim 36 wherein said script includes a sample rate.

38. The method of claim 36 wherein said script includes a number of sample regions.

39. The method of claim 36 wherein said script includes a radial distance.

40. The method of claim 36 wherein said script includes a time length.

41. The method of claim 36 wherein said script includes a centrifuge speed.

42. The method of claim 36 wherein said script includes a centrifuge duration.

43. The method of claim 36 wherein said script includes a n incubation duration.

44. A bio-disc data extraction system comprising a computer configured to control a bio-disc drive, an A/D card, and a storage device.

45. The data extraction system of claim 44 further including use of a successive approximation method.

46. The bio-disc data extraction system of claim 44 further comprising a set of assembly code wherein said set is used by said computer.

47. The bio-disc data extraction system of claim 46 wherein said set of assembly code omits an instruction wherein said instruction is not omitted by traditional compilers and wherein a set of parameters specific to said set of assembly code enables omission of said instruction without loss of correctness.

48. The bio-disc data extraction system of claim 47 wherein said instruction is an underflow flag check instruction that is unnecessary because it would follow a subtraction operation wherein due to constraints on the inputs of said subtraction operation an underflow cannot occur.

49. A bio-disc data extraction system, comprising:
a locator configured to determine one or more locations on said bio-disc;
a positioning unit configured to position a head of a bio-disc drive to read said one or more locations; and
a sampling unit configured to sample a signal generated by said head.

50. The bio-disc data extraction system of claim 49 wherein said locator further comprises:
one or more ditches obliterated on said bio-disc, each of said ditches having a leading edge and a trailing edge;
a separation between each of said ditches and each of said locations; and
a template configured to be placed over said bio-disc.

51. The bio-disc data extraction system of claim 50 wherein each of said locations is of a fixed rectangular size.

52. The bio-disc data extraction system of claim 50 wherein each of said locations is of a fixed circular size.

53. The bio-disc data extraction system of claim 50 wherein said separation between each of said ditches and each of said locations is fixed.

54. The bio-disc extraction system of claim 50 wherein one of said locations is preceded by a pair of ditches separated by a fixed distance.

55. The bio-disc extraction system of claim 50 wherein each of said ditches is divided into a grid.

56. The bio-disc extraction system of claim 55 wherein said grid has a plurality of vertical sections corresponding to a number of bits in a binary address encoding of said ditch.

57. The bio-disc extraction system of claim 55 wherein said grid has a plurality of horizontal sections corresponding to a number of bit combinations in said binary address encoding of said ditch.

58. The bio-disc extraction system of claim 55 wherein each section of said grid comprising one of said horizontal sections and one of said vertical sections is left un-burnt to indicate a binary zero.

59. The bio-disc extraction system of claim 55 wherein each section of said grid comprising one of said horizontal sections and one of said vertical sections is burnt to indicate a binary one.

60. The bio-disc data extraction system of claim 50 wherein said template has a set of markings that indicate radial distance.

61. The bio-disc data extraction system of claim 49 wherein said positioning unit comprises an input unit configured to obtain a radial distance.

62. The bio-disc data extraction system of claim 49 wherein said positioning unit comprises an input unit configured to obtain a time length.

63. The bio-disc extraction system of claim 49 wherein said positioning unit configured to position a head in association with said system further comprises control means to:
move said head of said bio-disc drive forward at fixed intervals until said head encounters said leading edge of a first of said ditches;
move said head of said bio-disc drive forward at fixed intervals when said head is over said first ditch;
move said head of said bio-disc drive backwards at fixed intervals until said head leaves said ditch until said head encounters said trailing edge of said first ditch;
move said head of said bio-disc drive forward at fixed intervals until said head encounters a first of said locations that is separated from said first ditch by a fixed distance; and
move said head of said bio-disc drive forward at fixed intervals until said head encounters a next location.

64. The bio-disc data extraction system of claim 49 wherein said sampling unit comprises:
an input unit configured to enter a decrease in strength of said signal when said signal enters one of said ditches;
an input unit configured to enter an increase in strength of said signal when said signal leaves one of said ditches;
an input unit configured to obtain a sample rate; and
an obtainer configured to obtain at said sample rate a value read by said head.

65. The bio-disc data extraction system of claim 49 wherein said locator comprises an input unit configured to obtain a first number of sample regions to be sampled.

66. The bio-disc data extraction system of claim 65 further comprising a de-interleaving unit configured to de-interleave a sampled signal into a second number of files.

67. The bio-disc data extraction system of claim 66 further comprising a storage device configured to store said second number of files, storage of said second number of files being performed concurrently with said sampling by said sampling unit.

68. The bio-disc data extraction system of claim 49 wherein said locator comprises an ordering unit configured to order a plurality of regions on said bio-disc.

69. The bio-disc data extraction system of claim 68 wherein said ordering unit comprises a determiner configured to identify a region marker having a distinguishing feature.

70. The bio-disc data extraction system of claim 69 wherein said region marker is a radial line.

71. The bio-disc data extraction system of claim 68 wherein said ordering unit comprises a determiner configured to determine a thinnest region marker.

72. The bio-disc data extraction system of claim 71 wherein said thinnest region marker is a radial line.

73. The bio-disc data extraction system of claim 68 wherein said ordering unit comprises a determiner configured to determine an order for said plurality of regions relative to a known location on said bio-disc.

74. The bio-disc data extraction system of claim 49 wherein said locator comprises a detection unit configured to detect a marking on an outer region of said bio-disc.

75. The bio-disc data extraction system of claim 74 wherein said marking is part of an encoding scheme.

76. The bio-disc data extraction system of claim 49 wherein a radial position of said head remains stationary while said sampling unit samples said signal.

77. A data extraction system for use with an optical analysis disc, said system comprising:
a positioning unit configured to position a head of a disc drive at a radial distance;
an input unit configured to obtain a desired centrifuge speed for said disc drive; and
a controller to direct spin of said disc at said desired centrifuge speed when said head is positioned at said radial distance.

78. The data extraction system of claim 77 further comprising a second input unit configured to obtain a duration so that said head is maintained at said radial distance for said duration.

79. An optical analysis disc data extraction system comprising a script that controls the behavior of said disc data extraction system.

80. The optical analysis disc data extraction system of claim 79 wherein said script includes a sample rate.

81. The optical analysis disc data extraction system of claim 79 wherein said script includes a number of sample regions.

82. The optical analysis disc data extraction system of claim 79 wherein said script includes a radial distance.

83. The optical analysis disc data extraction system of claim 79 wherein said script includes a time length.

84. The optical analysis disc data extraction system of claim 79 wherein said script includes a centrifuge speed.

85. The optical analysis disc data extraction system of claim 79 wherein said script includes a centrifuge duration.

86. The bio-disc data extraction system of claim 79 wherein said script includes an incubation duration.

87. A computer program product, comprising:
a computer usable medium having a computer readable program code embodied therein configured to extract data from an optical analysis disc; and
a computer readable code configured to cause a computer to control an optical analysis disc drive, an A/D card, and a storage device.

88. The computer program product of claim 87 wherein said computer readable program code implemented to extract said data from said optical analysis disc is a successive approximation program code.

89. The computer program product of claim 87 wherein said computer readable program code is derived from a set of assembly code.

90. The computer program product of claim 89 further including a set of parameters specific to said set of assembly code, said set of assembly code omitting an instruction that enables omission of said instruction without loss of correctness.

91. The computer program product of claim 90 wherein said instruction is an underflow flag check instruction that is unnecessary because it would follow a subtraction operation wherein due to constraints on the inputs of said subtraction operation an underflow cannot occur.

92. A computer program product, comprising:
a computer usable medium having a computer readable program code embodied therein configured to extract data from a optical analysis disc;
a first computer readable code configured to cause a computer to determine one or more locations on said optical analysis disc;
a second computer readable code configured to cause said computer to position a head of a disc drive to read said one or more locations; and
a third computer readable code configured to cause said computer to sample a signal generated by said head.

93. The computer program product of claim 92 wherein said first computer readable code comprises:
a fourth computer readable code configured to cause said computer to obtain a value generated from obliterating one or more ditches on said optical analysis disc, each of said ditches having a leading edge and a trailing edge;
a fifth computer readable code configured to cause said computer to obtain a value generated from separating each of said ditches from each of said locations; and
a sixth computer readable code configured to cause said computer to obtain a value generated from a template configured to be placed over said optical analysis disc.

94. The computer program product of claim 93 wherein each of said locations is of a fixed rectangular size.

95. The computer program product of claim 93 wherein each of said locations is of a fixed circular size.

96. The computer program product of claim 93 wherein a separation between each of said ditches and each of said locations is fixed.

97. The computer program product of claim 93 wherein one of said locations is preceded by a pair of ditches separated by a fixed distance.

98. The computer program product of claim 93 wherein each of said ditches is divided into a grid.

99. The computer program product of claim 98 wherein said grid has a plurality of vertical sections corresponding to a number of bits in a binary address encoding of said ditch.

100. The computer program product of claim 98 wherein said grid has a plurality of horizontal sections corresponding to a number of bit combinations in said binary address encoding of said ditch.

101. The computer program product of claim 98 wherein each section of said grid comprising one of said horizontal sections and one of said vertical sections is burnt to indicate a binary one.

102. The computer program product of claim 93 wherein each section of said grid comprising one of said horizontal sections and one of said vertical sections is left un-burnt to indicate a binary zero.

103. The computer program product of claim 93 wherein said template has a set of markings wherein said set of markings indicate radial distance.

104. The computer program product of claim 92 wherein said second computer readable code comprises a fourth computer readable code configured to cause said computer to obtain a radial distance.

105. The computer program product of claim 92 wherein said second computer readable code comprises a fourth computer readable code configured to cause said computer to obtain a time length.

106. The computer program product of claim 92 wherein said second computer readable code further comprises:
a fourth computer readable code configured to cause said computer to move said head of said disc drive forward at fixed intervals until said head encounters said leading edge of a first of said ditches;
a fifth computer readable code configured to cause said computer to move said head of said disc drive forward at fixed intervals when said head is over said first ditch;
a sixth computer readable code configured to cause said computer to move said head of said disc drive backwards at fixed intervals when said head leaves said ditch until said head encounters said trailing edge of said first ditch;
a seventh computer readable code configured to cause said computer to move said head of said disc drive forward at fixed intervals until said head encounters a first of said locations that is separated from said first ditch by a fixed distance; and
an eighth computer readable code configured to cause said computer to move said head of said disc drive forward at fixed intervals until said head encounters a next location.

107. The computer program product of claim 92 wherein said third computer readable code comprises:
a fourth computer readable code configured to cause said computer to obtain a sample rate; and
a fifth computer readable code configured to cause said computer to obtain at said sample rate a value read by said head.

108. The computer program product of claim 92 wherein said first computer readable code comprises a fourth computer readable code configured to cause said computer to obtain a first number of sample regions to be sampled.

109. The computer program product of claim 108 further comprising a fifth computer readable code configured to cause a computer to de-interleave a sampled signal into a second number of files.

110. The computer program product of claim 109 further comprising a sixth computer readable code configured to cause said computer to store said second number of files in a storage device wherein storage of said second number of files is performed concurrently with sampling of said signal.

111. The computer program product of claim 92 wherein said first computer readable comprises a fourth computer readable code configured to cause said computer to order a plurality of regions on said optical analysis disc.

112. The computer program product of claim 111 wherein said fourth computer readable code comprises a fifth computer readable code configured to cause said computer to determine a specific region marker.

113. The computer program product of claim 112 wherein said specific region marker is a radial line.

114. The computer program product of claim 111 wherein said fourth computer readable code comprises a fifth computer readable code configured to cause said computer to determine a thinnest region marker.

115. The computer program product of claim 114 wherein said thinnest region marker is a radial line.

116. The computer program product of claim 111 wherein said fourth computer readable code comprises a fifth computer readable code configured to cause said computer to determine an order for said plurality of regions relative to a known location on said optical analysis disc.

117. The computer program product of claim 92 wherein said first computer readable code comprises a fourth computer readable code configured to cause said computer to detect a marking on an outer region of said optical analysis disc.

118. The computer program product of claim 117 wherein said marking is part of an encoding scheme.

119. The computer program product of claim 92 wherein a radial position of said head remains stationary while said signal is sampled.

120. A computer program product, comprising:
a computer usable medium having computer readable program code embodied therein configured to extract data from an optical analysis disc;
computer readable code configured to cause a computer to obtain a desired centrifuge speed; and
computer readable code configured to cause a computer to position a head of a disc drive at a radial distance wherein said disc drive spins said optical disc at said desired centrifuge speed when said head is positioned at said radial distance.

121. The computer program product of claim 120 further comprising computer readable code configured to cause said computer to obtain a duration wherein said head is maintained at said radial distance for said duration.

122. A computer program product, comprising:
a computer usable medium having computer readable program code embodied therein configured to extract data from an optical analysis disc; and
computer readable code configured to cause a computer to execute a script that controls the behavior of an optical analysis disc data extraction system.

123. The computer program product of claim 122 wherein said script includes a sample rate.

124. The computer program product of claim 122 wherein said script includes a number of sample regions.

125. The computer program product of claim 122 wherein said script includes a radial distance.

126. The computer program product of claim 122 wherein said script includes a time length.

127. The computer program product of claim 122 wherein said script includes a centrifuge speed.

128. The computer program product of claim 122 wherein said script includes a centrifuge duration.

129. The computer program product of claim 122 wherein said script includes an incubation duration.

130. The method of claim 26 wherein specific region marker is a thickest region marker.

131. The bio-disc data extraction system of claim 66 wherein said first number is equal to said second number.

132. The bio-disc data extraction system of claim 69 wherein said distinguishing feature of said region marker is a predetermined thickness of said region marker.

133. The computer program product of claim 87 wherein said computer readable code is configured to cause a computer to concurrently control said optical analysis disc drive, said A/D card, and said storage device.

134. The computer program product of claim 92 wherein said first computer readable code, said second computer readable code, and said third computer readable code are integrated into a complete system code package that is loadable into a respective disc drive system to thereby enable said disc drive system to run said code package to analyze a sample associated with said optical analysis disc.

135. The computer program product of claim 93 wherein said fourth computer readable code, said fifth computer readable code, and said sixth computer readable code are integrated into a complete system code package that is loadable into a respective disc drive system to thereby enable said disc drive system to run said code package to analyze a sample associated with said optical analysis disc.

136. The method of claim 27 wherein specific region marker is a thickest region marker.

137. The computer program product of claim 93 wherein said first, second, third, fourth, fifth, and sixth computer readable codes are integrated into a complete system code package that is loadable into a respective disc drive system to thereby enable said disc drive system to run said code package to analyze a sample associated with said optical analysis disc.

138. The computer program product of claim 104 wherein said first, second, third, and fourth computer readable codes are integrated into a complete system code package that is loadable into a respective disc drive system to thereby enable said disc drive system to run said code package to analyze a sample associated with said optical analysis disc.

139. The computer program product of claim 105 wherein said first, second, third, and fourth computer readable codes are integrated into a complete system code package that is loadable into a respective disc drive system to thereby enable said disc drive system to run said code package to analyze a sample associated with said optical analysis disc.

140. The computer program product of claim 106 wherein said first, second, third, fourth, fifth, sixth, seventh, and eighth computer readable codes are integrated into a complete system code package that is loadable into a respective disc drive system to thereby enable said disc drive system to run said code package to analyze a sample associated with said optical analysis disc.

141. The computer program product of claim 107 wherein said first, second, third, fourth, and fifth computer readable codes are integrated into a complete system code package that is loadable into a respective disc drive system to thereby enable said disc drive system to run said code package to analyze a sample associated with said optical analysis disc.

142. The computer program product of claim 108 wherein said first, second, third, and fourth computer readable codes are integrated into a complete system code package that is loadable into a respective disc drive system to thereby enable said disc drive system to run said code package to analyze a sample associated with said optical analysis disc.

143. The computer program product of claim 109 wherein said first, second, third, fourth, and fifth computer readable codes are integrated into a complete system code package that is loadable into a respective disc drive system to thereby enable said disc drive system to run said code package to analyze a sample associated with said optical analysis disc.

144. The computer program product of claim 109 wherein said first number is equal to said second number.

145. The computer program product of claim 109 wherein said first, second, third, fourth, fifth, and sixth computer readable codes are integrated into a complete system code package that is loadable into a respective disc drive system to thereby enable said disc drive system to run said code package to analyze a sample associated with said optical analysis disc.

146. The computer program product of claim 111 wherein said first, second, third, and fourth computer readable codes are integrated into a complete system code package that is loadable into a respective disc drive system to thereby enable said disc drive system to run said code package to analyze a sample associated with said optical analysis disc.

147. The computer program product of claim 112 wherein said first, second, third, fourth, and fifth computer readable codes are integrated into a complete system code package that is loadable into a respective disc drive system to thereby enable said disc drive system to run said code package to analyze a sample associated with said optical analysis disc.

148. The computer program product of claim 112 wherein specific region marker includes a thickest region marker.

149. The computer program product of claim 114 wherein said first, second, third, fourth, and fifth computer readable codes are integrated into a complete system code package that is loadable into a respective disc drive system to thereby enable said disc drive system to run said code package to analyze a sample associated with said optical analysis disc.

150. The computer program product of claim 116 wherein said first, second, third, fourth, and fifth computer readable codes are integrated into a complete system code package that is loadable into a respective disc drive system to thereby enable said disc drive system to run said code package to analyze a sample associated with said optical analysis disc.

151. The computer program product of claim 117 wherein said first, second, third, and fourth computer readable codes are integrated into a complete system code package that is loadable into a respective disc drive system to thereby enable said disc drive system to run said code package to analyze a sample associated with said optical analysis disc.

152. The method of claim 4 wherein said instruction is an underflow flag check instruction associated with a subtraction operation wherein a first value involved in said subtraction operation is known to be larger than a second value involved in said subtraction operation wherein said second value is subtracted from said first value.

153. The bio-disc data extraction system of claim 47 wherein said instruction is an underflow flag check instruction associated with a subtraction operation wherein a first value involved in said subtraction operation is known to be larger than a second value involved in said subtraction operation wherein said second value is subtracted from said first value.

154. The computer program product of claim 90 wherein said instruction is an underflow flag check instruction associated with a subtraction operation wherein a first value involved in said subtraction operation is known to be larger than a second value involved in said subtraction operation wherein said second value is subtracted from said first value.
